SEQUENCE LISTING (63 sequences)

SEQ ID NO: 1 - protein, 5 aa, Artificial sequence
  PEPTIDE (1)..(5): Synthetic
GVGVP

SEQ ID NO: 2 - protein, 5 aa, Artificial sequence
  PEPTIDE (1)..(5): Synthetic
GVGIP

SEQ ID NO: 3 - protein, 10 aa, Artificial sequence
  VARIANT (7)..(7): the amino acid residue at position 7 is any naturally occurring amino acid or chemical modification thereof
GVGVPGXGVP

SEQ ID NO: 4 - protein, 5 aa, Artificial sequence
  VARIANT (2)..(2): the amino acid residue at position 2 is any naturally occurring amino acid or chemical modification thereof
GXGVP

SEQ ID NO: 5 - protein, 5 aa, Artificial sequence
  PEPTIDE (1)..(5): Synthetic
VPGVG

SEQ ID NO: 6 - protein, 5 aa, Artificial sequence
  PEPTIDE (1)..(5): Synthetic
IPGVG

SEQ ID NO: 7 - protein, 6 aa, Artificial sequence
  PEPTIDE (1)..(6): Synthetic
APGVGV

SEQ ID NO: 8 - protein, 4 aa, Artificial sequence
  PEPTIDE (1)..(4): Synthetic
VPGG

SEQ ID NO: 9 - protein, 4 aa, Artificial sequence
  VARIANT (3)..(3): the amino acid residue at position 3 is V, F or A
GGXP

SEQ ID NO: 10 - protein, 5 aa, Artificial sequence
  VARIANT (2)..(4): the amino acid residue at position 2 is V, E, F, Y, K, I or A; the amino acid residue at position 4 is V, E, F or I
GXGXP

SEQ ID NO: 11 - protein, 30 aa, Artificial sequence
  VARIANT (27)..(27): the amino acid residue at position 27 is E or K
GFGFPGFGVPGAGVPGFGFPGIGVPGXGVP

SEQ ID NO: 12 - protein, 5 aa, Artificial sequence
  VARIANT (2)..(2): the amino acid residue at position 2 is any amino acid residue modified to have an electroresponsive side chain
GXGVP

SEQ ID NO: 13 - protein, 30 aa, Artificial sequence
  PEPTIDE (1)..(30): Synthetic
GAGFPGFGVPGAGVPGIGFPGFGVPGKGVP

SEQ ID NO: 14 - DNA, 203 bp, Artificial sequence
  misc_feature (1)..(203): Synthetic
gaggatccaggctttggtttcccgggtttcggcgttccgggcgctggtgtaccgggtttcggctttccgggtatcggtgttccgggcgaaggtgtgccaggattcggcttcccgggctttggtgttccgggtgcaggcgtaccgggtttcggtttcccgggcattggcgttccgggtgaaggtgtaccaggctttggatccag

SEQ ID NO: 15 - protein, 6 aa, Artificial sequence
  PEPTIDE (1)..(6): Synthetic
GRGDSP

SEQ ID NO: 16 - protein, 6 aa, Artificial sequence
  PEPTIDE (1)..(6): Synthetic
RGYSLG

SEQ ID NO: 17 - protein, 30 aa, Artificial sequence
  PEPTIDE (1)..(30): Synthetic
GEGVPGFGFPGFGVPGAGVPGFGFPGIGVP

SEQ ID NO: 18 - protein, 30 aa, Artificial sequence
  PEPTIDE (1)..(30): Synthetic
GKGVPGAGFPGFGVPGAGVPGIGFPGFGVP

SEQ ID NO: 19 - protein, 10 aa, Artificial sequence
  PEPTIDE (1)..(10): Synthetic
GVGVPGVGPP

SEQ ID NO: 20 - protein, 35 aa, Artificial sequence
  REPEAT (1)..(30): Synthetic
GVGVPGVGFPGEGFPGVGVPGVGFPGFGFPGVGVP

SEQ ID NO: 21 - protein, 35 aa, Artificial sequence
  REPEAT (1)..(30): Synthetic
GVGVPGVGFPGEGFPGVGVPGVGFPGVGFPGVGVP

SEQ ID NO: 22 - protein, 35 aa, Artificial sequence
  REPEAT (1)..(30): Synthetic
GVGVPGVGVPGEGVPGVGVPGVGFPGFGFPGVGVP

SEQ ID NO: 23 - protein, 35 aa, Artificial sequence
  REPEAT (1)..(30): Synthetic
GVGVPGVGFPGEGFPGVGVPGVGVPGVGVPGVGVP

SEQ ID NO: 24 - protein, 35 aa, Artificial sequence
  REPEAT (1)..(30): Synthetic
GVGVPGVGVPGEGVPGVGVPGVGVPGVGVPGVGVP

SEQ ID NO: 25 - protein, 65 aa, Artificial sequence
  REPEAT (1)..(60): Synthetic
GVGIPGFGEPGEGFPGVGVPGFGFPGFGIPGVGIPGFGEPGEGFPGVGVPGFGFPGFGIPGVGVP

SEQ ID NO: 26 - protein, 35 aa, Artificial sequence
  REPEAT (1)..(30): Synthetic
GVGVPGVGFPGKGFPGVGVPGVGFPGFGFPGVGVP

SEQ ID NO: 27 - protein, 35 aa, Artificial sequence
  REPEAT (1)..(30): Synthetic
GVGVPGVGFPGKGFPGVGVPGVGFPGVGFPGVGVP

SEQ ID NO: 28 - protein, 35 aa, Artificial sequence
  REPEAT (1)..(30): Synthetic
GVGVPGVGVPGKGVPGVGVPGVGFPGFGFPGVGVP

SEQ ID NO: 29 - protein, 35 aa, Artificial sequence
  REPEAT (1)..(30): Synthetic
GVGVPGVGFPGKGFPGVGVPGVGVPGVGVPGVGVP

SEQ ID NO: 30 - protein, 35 aa, Artificial sequence
  REPEAT (1)..(30): Synthetic
GVGVPGVGVPGKGVPGVGVPGVGVPGVGVPGVGVP

SEQ ID NO: 31 - protein, 35 aa, Artificial sequence
  REPEAT (1)..(30): Synthetic
GVGVPGVGFPGEGFPGVGVPGVGFPGKGVPGVGVP

SEQ ID NO: 32 - protein, 35 aa, Artificial sequence
  REPEAT (1)..(30): Synthetic
GVGVPGVGFPGEGFPGVGVPGVGVPGKGVPGVGVP

SEQ ID NO: 33 - protein, 45 aa, Artificial sequence
  REPEAT (1)..(45): Synthetic
GVGIPGEGIPGVGIPGVGIPGEGIPGVGIPGVGIPGEGIPGVGIP

SEQ ID NO: 34 - protein, 30 aa, Artificial sequence
  REPEAT (1)..(30): Synthetic
GVGIPGVGIPGEGIPGVGIPGVGIPGVGIP

SEQ ID NO: 35 - protein, 30 aa, Artificial sequence
  REPEAT (1)..(30): Synthetic
GEGIPGVGIPGEGIPGVGIPGVGIPGVGIP

SEQ ID NO: 36 - protein, 45 aa, Artificial sequence
  REPEAT (1)..(45): Synthetic
GVGIPGKGIPGVGIPGVGIPGKGIPGVGIPGVGIPGKGIPGVGIP

SEQ ID NO: 37 - protein, 30 aa, Artificial sequence
  REPEAT (1)..(30): Synthetic
GVGIPGVGIPGKGIPGVGIPGVGIPGVGIP

SEQ ID NO: 38 - protein, 30 aa, Artificial sequence
  REPEAT (1)..(30): Synthetic
GKGIPGVGIPGKGIPGVGIPGVGIPGVGIP

SEQ ID NO: 39 - protein, 110 aa, Artificial sequence
  REPEAT (1)..(110): Synthetic
GVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGYGIP

SEQ ID NO: 40 - protein, 110 aa, Artificial sequence; identical to SEQ ID NO: 39
  VARIANT (107)..(107): Residue at position 107 is associated with an SO4 ion

SEQ ID NO: 41 - protein, 60 aa, Artificial sequence
  REPEAT (1)..(60): Synthetic
GVGIPGVGIPGVGIPGVGIPGVGIPGVGIPGVGIPGVGIPGVGIPGVGIPGVGIPGYGIP

SEQ ID NO: 42 - protein, 60 aa, Artificial sequence; identical to SEQ ID NO: 41
  REPEAT (1)..(60): Synthetic

SEQ ID NO: 43 - protein, 45 aa, Artificial sequence
  REPEAT (1)..(45): Synthetic
GVGIPGVGIPGVGIPGVGIPGVGIPGVGIPGVGIPGVGIPGYGIP

SEQ ID NO: 44 - protein, 45 aa, Artificial sequence; identical to SEQ ID NO: 43
  REPEAT (1)..(45): Synthetic

SEQ ID NO: 45 - protein, 30 aa, Artificial sequence
  REPEAT (1)..(30): Synthetic
GVGIPGVGIPGVGIPGVGIPGVGIPGYGIP

SEQ ID NO: 46 - protein, 30 aa, Artificial sequence; identical to SEQ ID NO: 45
  REPEAT (1)..(30): Synthetic

SEQ ID NO: 47 - protein, 15 aa, Artificial sequence
  REPEAT (1)..(15): Synthetic
GVGIPGVGIPGYGIP

SEQ ID NO: 48 - protein, 15 aa, Artificial sequence
  REPEAT (1)..(15): Synthetic
GVGIPGVGIPGYGIP

SEQ ID NO: 49 - DNA, 66 bp, Artificial sequence
  misc_feature (1)..(66): Synthetic
ggaagtggttgctgcaactccgacttctctgctgatctcctgggacgctccagcagttaccgtacg

SEQ ID NO: 50 - DNA, 66 bp, Artificial sequence
  misc_feature (1)..(66): Synthetic
gaaccggagagttgccaccagtctcgccgtaggtgatgcgatagtaacgtacggtaactgctggag

SEQ ID NO: 51 - DNA, 67 bp, Artificial sequence
  misc_feature (1)..(67): Synthetic
gaggatccaggcgttggcattccggtttctgacgtaccgcgtgatctggaagtggttgctgcaactc

SEQ ID NO: 52 - DNA, 64 bp, Artificial sequence
  misc_feature (1)..(64): Synthetic
ctggatccaacgcctgggatacctacacccatggtctgaattcctgaaccggagagttgccacc

SEQ ID NO: 53 - DNA, 73 bp, Artificial sequence
  misc_feature (1)..(73): Synthetic
ccaggcagcaagtccactgcgaccatttctggtctgaaaccgggtgttgactacaccatcactgtgtacgctg

SEQ ID NO: 54 - DNA, 73 bp, Artificial sequence
  misc_feature (1)..(73): Synthetic
gtagttaatggagatcggtttgctggaagcacccagagaatagccacgagttacagcgtacacagtgatggtg

SEQ ID NO: 55 - DNA, 73 bp, Artificial sequence
  misc_feature (1)..(73): Synthetic
gaggatccaggcgttggcattccgtaagctttctcgagagagaattcaccgtaccaggcagcaagtccactgc

SEQ ID NO: 56 - DNA, 72 bp, Artificial sequence
  misc_feature (1)..(72): Synthetic
ctggatccaacgcctgggatacctacacccggtttgtcgatttcggtacggtagttaatggagatcggtttg

SEQ ID NO: 57 - protein, 99 aa, Artificial sequence
  PEPTIDE (1)..(99): Synthetic
VSDVPRDLEVVAATPTSLLISWDAPAVTVRYYRITYGETGGNSPVQEFTVPGSKSTATISGLKPGVDYTITVYAVTRGYSLGASSKPISINYRTEIDKP

SEQ ID NO: 58 - DNA, 205 bp, Artificial sequence
  misc_feature (1)..(205): Synthetic
gaggatccaggcgttggcattccggtttctgacgtaccgcgtgatctggaagtggttgctgcaactccgacttctctgctgatctcctgggacgctccagcagttaccgtacgttactatcgcatcacctacggcgagactggtggcaactctccggttcaggaattcagaccatgggtgtaggtatcccaggcgttggatccag

SEQ ID NO: 59 - protein, 62 aa, Artificial sequence
  PEPTIDE (1)..(62): Synthetic
PGVGIPVSDVPRDLEVVAATPTSLLISWDAPAVTVRYYRITYGETGGNSPVQEFMGVGIPGV

SEQ ID NO: 60 - DNA, 205 bp, Artificial sequence
  misc_feature (1)..(205): Synthetic
ctcctaggtccgcaaccgtaaggccaaagactgcatggcgcactagaccttcaccaacgacgttgaggctgaagagacgactagaggaccctgcgaggtcgtcaatggcatgcaatgatagcgtagtggatgccgctctgaccaccgttgagaggccaagtccttaagtctggtacccacatccatagggtccgcaacctaggtc

SEQ ID NO: 61 - DNA, 229 bp, Artificial sequence
  misc_feature (1)..(229): Synthetic
gaggatccaggcgttggcattccgtaagctttctcgagagagaattcaccgtaccaggcagcaagtccactgcgaccatttctggtctgaaaccgggtgttgactacaccatcactgtgtacgctgtaactcgtggctattctctgggtgcttccagcaaaccgatctccattaactaccgtaccgaaatcgacaaaccgggtgtaggtatcccaggcgttggatccag

SEQ ID NO: 62 - protein, 66 aa, Artificial sequence
  PEPTIDE (1)..(66): Synthetic
PGVGIPEFTVPGSKSTATISGLKPGVDYTITVYAVTRGYSLGASSKPISINYRTEIDKPGVGIPGV

SEQ ID NO: 63 - DNA, 229 bp, Artificial sequence
  misc_feature (1)..(229): Synthetic
ctcctaggtccgcaaccgtaaggcattcgaaagagctctctcttaagtggcatggtccgtcgttcaggtgacgctggtaaagaccagactttggcccacaactgatgtggtagtgacacatgcgacattgagcaccgataagagacccacgaaggtcgtttggctagaggtaattgatggcatggctttagctgtttggcccacatccatagggtccgcaacctaggtc

(19) United States
(12) Patent Application Publication
(10) Pub. No.: US 2002/0068304 A1
(43) Pub. Date: Jun. 6, 2002

(54) Bioelastomer nanomachines and biosensors

(76) Inventor: Dan W. Urry, Birmingham, AL (US)

Correspondence Address: COOLEY GODWARD, LLP, 3000 EL CAMINO REAL, 5 PALO ALTO SQUARE, PALO ALTO, CA, 94306, US

(21) Appl. No.: 09/888,260

(22) Filed: Jun. 21, 2001

Related U.S. Application Data
(60) Provisional application No. 60/213,364, filed on Jun. 23, 2000.

(51) Int. Cl. G01N 33/53; F02B 1/00

(52) U.S. Cl. 435/7.1; 60/721

(57) ABSTRACT

Bioelastomers, having repeating peptide monomeric units selected from the group consisting of bioelastic nonapeptides, pentapeptides and tetrapeptides, are used to produce nanomachines and biosensors.

### CROSS REFERENCE TO RELATED APPLICATIONS

This application claims the benefit of U.S. Provisional Application No. 60/213,364, filed on Jun. 23, 2000.

### TECHNICAL FIELD

The present invention relates to the design of bioelastomers suitable for use as nanomachines and biosensors.

### BACKGROUND

Elastic protein-based polymers or bioelastic polymers, referred to herein as “bioelastomers”, are often described as comprising repeating peptide monomeric units selected from the group consisting of nonapeptide, pentapeptide and tetrapeptide monomeric units, where the monomeric units form a series of β-turns separated by dynamic bridging segments suspended between said β-turns. These bioelastomers are well suited for a wide variety of uses since their defined elements of structure allows them to be designed and synthesized with chosen physical properties, rather than having to rely on the less controllable properties of materials prepared from random chain network elastomers such as natural rubber and synthetic analogues.

At the millimeter scale, bioelastomers have been designed to perform many forms of pair-wise free energy transduction involving the six intensive variables of mechanical force, temperature, pressure, chemical potential, electrochemical potential, and electromagnetic radiation. It would be desirable to design bioelastomers such that the pair-wise energy conversions capable of the performance of mechanical work can be performed at the nanometer scale using atomic force microscopy (“AFM”).

This effort is to achieve a thousand-fold decrease in scale from the generally considered micro-electro-mechanical systems (“MEMS”) and a million fold step in scale from the visually observable energy converting cross-linked elastomeric matrices that have previously been developed. The timing is appropriate for a number of reasons: (1) it is now possible to use AFM to perform single chain force-extension studies as previously demonstrated with DNA, polysaccharides, proteins such as titin, and polyethylene glycol; (2) single chain force-extension curves are now available for two compositions of elastic protein-based polymers; (3) the free energy transductions involving the performance of mechanical work, which has previously been demonstrated at the macroscopic (millimeter) scale using designed bioelastomers, now have the possibility of being performed at the nanometer scale; and (4) the results would represent remarkable increases in sensitivity, thereby expanding the potential applications of these biomolecular machines.

The present invention provides compositions and methods for meeting those needs by use of bioelastomers. By the methods and compositions described herein: response times can be as short as the millisecond range with the potential to become shorter; the measurable length changes are at the one nanometer scale or less, and the measurable force changes are in the (10 picoNewton (pN) range. In the role of biosensor there is now the possibility of detecting single molecular events. For example, the binding of a single nerve gas molecule, or other important analyte, to a site on a hydrophobically folded globular protein in series with an appropriate elastomeric polypeptide now has the potential to be detected by a change in the force-length profile for such an elastic nanofilament construct.

### SUMMARY OF THE INVENTION

One aspect of the invention relates to designing bioelastomers that find utility as nanomachines.

Another aspect of the invention pertains to nanomachines comprising a bioelastomer having repeating peptide monomeric units selected from the group consisting of nonapeptide, pentapeptide and tetrapeptide monomeric units, wherein said monomeric units form a series of β-turns separated by dynamic bridging segments suspended between said β-turns.

Yet another aspect of the invention relates to nanomachines comprising a bioelastomer which is in the form of nanoparticles or in the form of multi-stranded nanofilaments.

Still another element of the invention involves a single globular domain containing a binding site in series with the bioelastomer chains, wherein binding of an analyte at the binding site causes the hydrophobically folded globular domain to unfold at a different force level (higher or lower) for comparable rates of extension.

### DESCRIPTION OF THE FIGURES

FIG. 1 illustrates the pair-wise energy conversions of which the Tₜ-based molecular machines of the invention are capable. This presents the (Tₜ hydrophobic paradigm for protein folding and function.

FIGS. 2A-2C depict single-chain force-extension curves.

FIG. 2A depicts the curves for diluted and for very diluted C(GVGVP)ₙC in steps of 251 with 502 being the most reasonable specific chain length, where (GVGVP)ₙ is SEQ ID NO:1, n is an integer from 251 to 1004, and C are cysteinyl residues at the ends of the single chain.

FIG. 2B depicts the curve for very diluted (GVGIP)ₙ(SEQ ID NO:2 where n is an integer from 260 to 1280), while

FIG. 2C depicts the curve for diluted (GVGIP)ₙ, where there is hydrophobic interchain interaction

FIGS. 3A-3G depict the molecular structure of poly(GVGVP) (SEQ ID NO:1), based upon NMR, electron microscopy, computations and crystal structure data.

FIG. 3A depicts the schematic representation of an extended chain with the Pro-Gly inserted β-turn. The detailed β-turn, as confirmed by the crystal structure of the cyclic analogue, cyclo(GVGVP)₃ (SEQ ID NO:1; n-3), is shown in FIG. 3B.

FIG. 3C is a schematic representation of a helix with dimensions of a polypentapeptide bioelastomer P-spiral.

FIG. 3D is a schematic band representation of theβ-spiral showing the β-turns as spacers between the turns of the helical spiral.

FIGS. 3E and 3F show the detailed bond representation in stereo pair showing the β-turns and suspended segments between b-turns, and the space within the β-spiral occupied by water. FIG. 3E shows the side view in stereo pair, while FIG. 3F depicts the stereo pair giving the axis view. The fundamental unit is considered to be the twisted filament of β-spirals, shown in FIG. 3G.

FIGS. 4 and 4B are schematic representations of a twisted filament comprised of three strands of (GVGVP)ₙ(SEQ ID NO:1; n—251).

FIG. 4A shows the Kemp triacid holding the tree strands at each end and with cysteinyl residues added at both ends of each strand to provide for binding to the gold coated surfaces involved in the atomic force microscopy experiment. In FIG. 4B, the “R” represents an amino functions such as a series of lysine residues or a carboxy function such as a series of glutamic acid residues.

FIGS. 5A-5C depict how the control of the amino acid sequence directs the triple-stranded twisted filament formation.

FIGS. 5A and 5B show the folded P-spiral structure separating oil-like R-groups from vinegar-like R-groups. FIG. 5A illustrates oil-like residues on one side and vinegar-like residues on the other, while FIG. 5B illustrates the oil-like side of he twisted filament.

FIG. 5C shows the associated folded chains burying the oil-like R-groups away from water (seen end on), where the oil-like residues are depicted in the center, separated from the vinegar-like residues and from water on the periphery.

FIG. 6 depicts a hypothetical force-extension profile showing the effect of analyte interaction with a singular globular protein (“GP”). Curve A corresponds to a single strand of bioelastomer (“BE”). Curve B corresponds to the hypothetical curve for BE-GP-BE in which the globular domain is comprised of two hydrophobic folding domains. Curve C corresponds to Curve B plus the analyte having bound to one of the two hydrophobically folded domains of GP causing one to unfold.

FIG. 7 is the acoustic absorption data of 20 Mrad γ-irradiation cross-linked (GVGIP)₂₆₀ (SEQ ID NO:2 where n=260) showing a frequency range limited mechanical resonance to increase as the temperature is raised from below to above the onset of the inverse temperature transition of hydrophobic folding and assembly. Two overlapping peaks are observed, which, by analogy to optical absorption of helical structures, could be components of the mechanical resonance resolved parallel and perpendicular to the helical (β-spiral) axis. This mechanical resonance provides an opportunity to monitor the change in state, folded or unfolded, as induced by another intensive variable of the free energy acting on another functionality of the polymer.

### DESCRIPTION OF SPECIFIC EMBODIMENTS

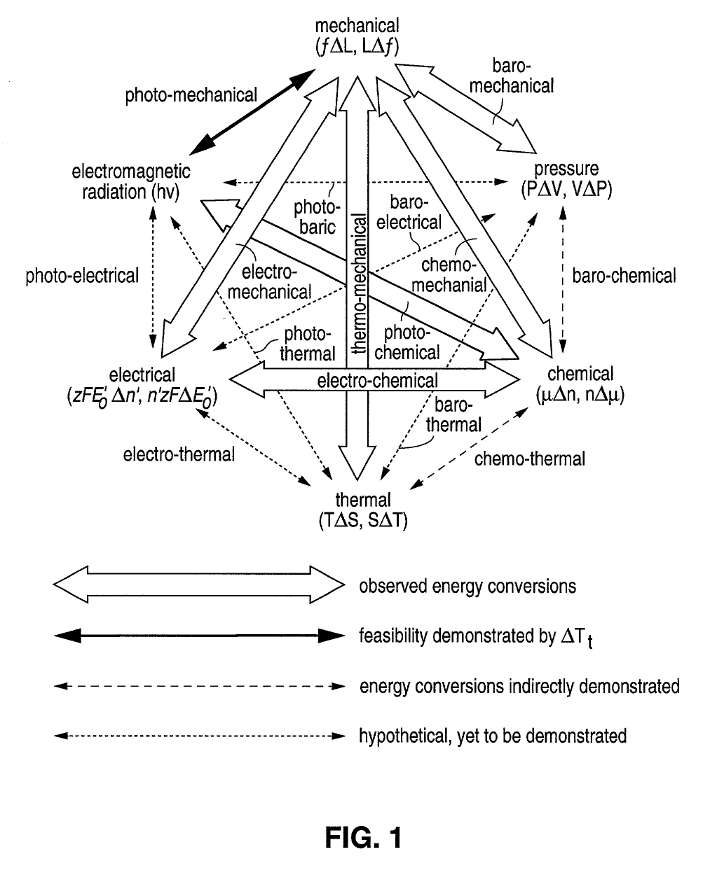

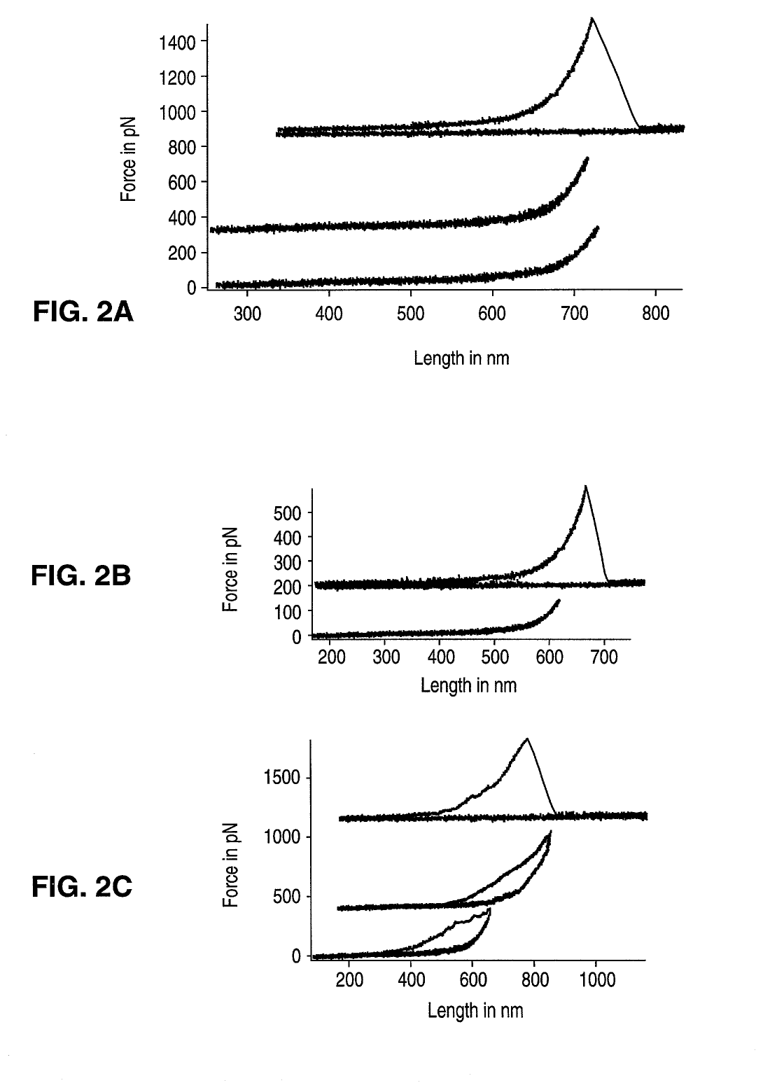

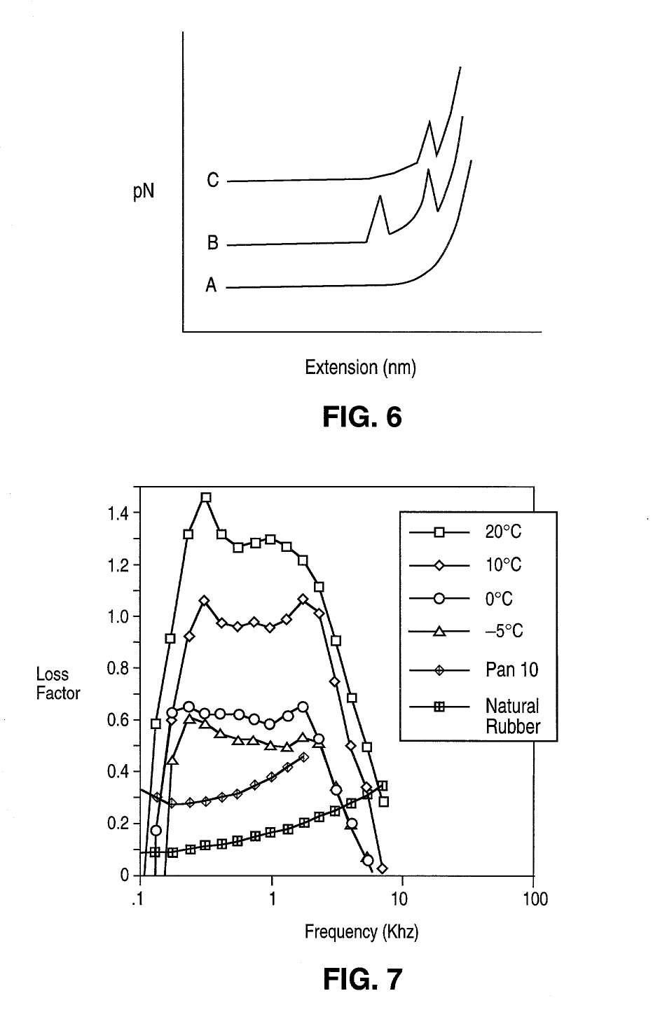

The invention pertains to bioelastomers useful as nanomachines and biosensors having dimensions of approximately 5 mn in width and hundreds of nm in length, along with methods for designing such bioelastomers. In general, the nanomachines and biosensors of the invention comprise a bioelastomer, having repeating peptide monomeric units selected from the group consisting of bioelastic nonapeptides, pentapeptides and tetrapeptides. The bioelastomer can be in the form of nanoparticles or multi-stranded nanofilaments. These different multi-stranded nanofilaments of bioelastomers can be designed to function as nanochemomechanical systems (“NCMS”), as nanoelectromechanical systems (“NEMS”), nanobaromechanical systems, nanothermomechanical systems and nanophotomechanical systems, all of which are described in FIG. 1; as well as nanoelectromagnetic radiation driven mechanical systems (“NEMRDMS”) and as biosensors for detecting the presence of chemical species, e.g., nerve gas, TNT, DNT, etc. Binding polar analytes, for example, can raise the temperature, Tₜ, of a hydrophobic folding transition from below to above the operating temperature with consequences of unfolding and atomic force microscopy-measurable changes in force-length profiles, or at fixed length, changes in the intensity of the parallel component of the acoustic absorption. In addition, instead of multi-stranded twisted nanofilaments, the nanomachine or biosensor can also be formed by a single chain folding back on itself with the forced unfolding causing either a peaking or an increase in force of the force-extension profile, as seen in FIG. 2C. Additionally, the globular protein component could contain enzyme recognition sites, e.g., kinase recognition sites, which on phosphorylation would cause the hydrophobically folded globular domain to unfold at a lower force level in the manner indicated in FIG. 6.

The invention relates to designing bioelastomers such that several pair-wise energy conversions can be performed at the nanometer scale using atomic force microscopy (“AFM”). This is accomplished by assembling one or more identical protein-based polymer strands, approaching 10,000, more typically about 2000 residues in length, using di-, tri-, and tetra-acids at each end, such as adipic acid (hexandioic acid), Kemp triacid (shown in FIG. 4A), and ethylenediaminetetraacetic acid (“EDTA”). The resulting single or multi-stranded, twisted nanofilaments are then strung between a cantilever and a substrate using cysteinyl sulfurs at the multi-acid ends for attachment. Typically, attachment is between the tip of the cantilever and the surface of the substrate, both of which are preferably coated with a suitable material such as gold. Other means of attachment to the cantilever and substrate surfaces can be anticipated, using other functional groups such as the amino, carboxyl functions in place of the SH moieties of cysteinyl residues.

In one embodiment of the invention, the cantilever senses the vibrational energy absorbed by the bioelastomer. This is illustrated in FIG. 7.

When using nanomachines based solely on bioelastomers, altering one or a few functional group(s) each hundred residues can completely unfold a totally contracted multi-stranded filament, changing length at a given force by hundreds of nanometers. Examples would be the phosphorylation of a single serine residue per every 30-500 amino acid residues, typically about every 100 residues; ionizing two carboxyl side chains per every 20-200 amino acid residues, typically about every 100 residues; or the oxidation of two attached redox functions, e.g., N-methyl nicotinamides, per every 20-200 amino acid residues, typically about every 100 residues. With 10 pN and nm sensitivities, AFM-derived single-chain force-extension curves can demonstrate the force-length profiles for unfolding individual hydrophobically-folded globular proteins. Using AFM in combination with bioelastomers in series with a single hydrophobically-folded globular protein, there is the ultimate potential, selective detection of binding a single molecule to a specific site on the globular protein by means of altering its force profile for unfolding.

These elastic protein-based polymers are capable of sensing energy inputs or of providing energy outputs over a remarkably wide range of frequencies of the electromagnetic spectrum from the high frequency range of approximately 10¹⁵ cycles per second to frequencies down to 10 Hz (cycles per second). Typical frequencies would be within the range of 10-10⁵, more typically 10²-10⁴ cycles per second. At the high frequency end are the diverse set of chromophores that on absorption of light change their hydrophobicity. At an intermediate range of 5 GHz in the dielectric relaxation spectrum, there is the absorption of hydrophobic hydration. Also at an intermediate range of 5 MHz in the dielectric relaxation spectrum, there is a structural resonance of the hydrophobically folded and assembled state. And at the low frequency range in both the dielectric relaxation and the sound absorption spectrum near 1 kHz, there is a mechanical resonance. It should be noted that the mechanical resonance of FIG. 7 near 1 kHz would best be considered as a means of introducing the intensive variable of mechanical force. Each of these absorption processes can be a means of energy input into the polymeric system that can change the state of the system or alternatively can represent a change in the energy output due to a change in the system resulting from another energy input. For example, in the AFM single chain (or single twisted filament) experiment at a fixed extension a change in force due to a change in folding would also be accompanied by a change in the intensity of the mechanical resonance as seen in FIG. 7 where the intensive variable is temperature. There are various ways to detect changes in the state of folding of the twisted filament resulting from the introduction of any of the intensive variables of the free energy (e.g., given in FIG. 1) to which the polymer is responsive. For example, changes in the intensity of the parallel component of the mechanical resonance of FIG. 7 would provide a means of detecting a change in a free energy input that changed the folded state of the polymer, and the combination of the energy input and its detection by a change in the mechanical resonance would constitute a free energy transducer.

Before further describing the methods and compositions of the invention, it is important to have an understanding of macroscopic bioelastomer molecular machines, a subject reviewed in Urry , J. Phys. Chem. B, 101:1 1007-11028, 1997. Certain key areas will be summarized herein: the (ΔTₜ-mechanism (controlling the temperature of hydrophobic folding and assembly in amphiphilic polymers such as proteins), free energy transduction by the (ΔTₜ-mechanism, and the physical basis for the (Tₜ-mechanism (an apolar-polar repulsive free energy of hydration). In addition, the AFM-single-chain force extension curves of natural elastic proteins also provide helpful information for understanding the scope of the invention. This previously demonstrated macroscopic free energy transduction can become nanoscopic free energy transduction by utilizing AFM. The data would be in the form of single nanofilament force-extension curves. The result can be nanomachines, for example, nanoelectromechanical systems.

### ΔTₜ-Mechanism (Controlling the Temperature of Hydrophobic Folding and Assembly in Amphiphilic Polymers such as Proteins and Protein-based Polymers

A. Definition of Tₜ and the Inverse Temperature Transition

The bioelastomers of interest here have a balance of polar (e.g., charged) and apolar (hydrophobic) moieties such that they are soluble at low temperature and hydrophobically fold and assemble as the temperature is raised. The temperature for the onset of this hydrophobic aggregation is designated as Tₜ. Cyclic analogues have been seen to crystallize on raising the temperature and to re-dissolve, once again to become randomly dispersed in solution on lowering the temperature. Furthermore macroscopically, the linear high molecular weight polymers undergo a phase separation and at the molecular level are found to have assembled in the formation of associated twisted filaments as seen in negatively stained micrographs.

As the order of the polypeptide part of the system clearly increases on raising the temperature, this has been called an inverse temperature transition. The term has analogy to cold denaturation of proteins and to the lower critical solution temperature (“LCST”) of amphiphilic petroleum-based polymers, but the term inverse temperature transition is used because it is more general and informative than the other terms. For example, the term LCST is not relevant to the hydrophobic folding of an individual globular protein, nor is cold denaturation relevant to the dissolution of a crystal without significant change in conformation. Nonetheless in all cases the same process applies, a change in hydrophobic interaction resulting in a change in entropy of the polymer component of the system. There is another important distinction between the inverse temperature transition of protein-based polymers and the LCST of petroleum-based polymers such as poly(N-isopropylacrylamide) (“PNIPAM”). For PNIPAM the phase separation occurs at 34° C., but the transition is to a disordered state of about 30% water by weight. (See Grinberg, et al., “Studies of the Thermal Volume Transition of Poly(N-isopropylacrylamide) Hydrogels by High Sensitivity Differential Scanning Microcalorimetry. 1. Dynamic Effects.” in Macromolecules 32:1471-1475, 1999). On the other hand for protein-based polymers of concern here, such as poly(GVGVP) (SEQ ID NO:1), the phase transition begins at 25° C. with formation of the structured β-spiral state that is 63% water by weight. Only after temperatures greater than 60° C. have been reached does the β-spiral state denature to form the disordered state of about 30% water by weight.

B. The Many Means of Controlling the Value of Tₜ

The value of Tₜ depends on several factors, including the following: i) polymer concentration; ii) polymer chain length; iii) polymer amino acid composition; iv) concentration of salts, e.g., the Hofineister (Lyotropic) Series; v) organic solutes and solvents; vi) polymer side-chain ionization; vii) chemical modification of polymer side chains, e.g., phosphorylation, nitration, sulfation, and glycosylation; viii) pressure (special role of aromatic residues); ix) redox state of prosthetic group attached to polymer, e.g., N-methyl nicotinamide (“NMeN”), nicotinamide adenine dinucleotide (“NAD”), and flavin adenine dinucleotide (“FAD”); x) the absorption of light by a prosthetic group attached to the polymer, e.g., azobenzene and cinnamide, or of any other appropriate frequency of the electromagnetic spectrum from 10 Hz to 10¹⁵ cycles per second; and xi) side chain charge neutralization by ion pairing, i.e., cation neutralization of anionic side chain, anionic neutralization of cationic side chain, and ion-pairing within and between chains. The foregoing enumeration lists the means whereby the value of Tₜcan be controlled to perform various kinds of free energy transduction. These dependencies of the temperature, Tₜ, of the inverse temperature transition are set forth in Urry, J. Phys. Chem. B, 101:11007-11028, 1997.

C. The Tₜ-based Hydrophobicity Scale

One of the particularly useful means of varying the value of Tₜ listed above is the effect of amino acid composition and chemical modification of side chains. This data has been compiled as a Tₜ-based hydrophobicity scale of particular importance to protein engineering, as shown in Tables 1A and 1B, which provide the Tₜ-based hydrophobicity scales for protein engineering for poly{ƒᵥ(GVGVP), ƒₓ(GX¹GVP)} (SEQ ID NO:3, where X¹ is any naturally occurring amino acid or chemical modification thereof) and also includes values of (ΔHₜ and ΔSₜ for the inverse temperature transition.

TABLE 1A
Tₜ-based Hydrophobicity Scale for the Naturally Occurring Amino Acid Residues

| | | | | ΔH^{e}, | ΔS^{e}, |
|---|---|---|---|---|---|
| | | Abbre- | | kcal/mol^{d} | cal/mol-K^{d} |
| Residue | R Group | viation | Tₜ^{a} | ±0.05 | ±0.05 |
| Tryptophan | —CH₂-indolyl | Trp (W) | −90° C. | 2.10 | 7.37 |
| Tyrosine | —CH₂-phenol | Tyr (Y) | −55° C. | 1.87 | 6.32 |
| Phenylalanine | —CH₂-phenyl | Phe (F) | −30° C. | 1.93 | 6.61 |
| Histidine | —CH₂-imidazolyl | His (H) | −10° C. | — | — |
| Proline (calc.)^{b} | —(CH₂)₃— | Pro (P) | (−8° C.) | — | — |
| Leucine | —CH₂—CH(CH₃)₂ | Leu (L) | 50° C. | 1.51 | 5.03 |
| Isoleucine | —CH(CH₃)CH₂CH₃ | Ile (I) | 10° C. | 1.43 | 4.60 |
| Methionine | —(CH₂)₂SCH₃ | Met (M) | 20° C. | 1.00 | 3.29 |
| Valine | —CH(CH₃)₂ | Val (V) | 24° C. | 1.20 | 3.90 |
| Histidine | —CH₂-imidazolyl⁺ | His⁺ (H⁺) | 30° C. | — | — |
| Glutamic Acid | —(CH₂)₂COOH | Glu (E) | 30° C. | 0.96 | 3.14 |
| Cysteine | —CH₂SH | Cys (C) | 30° C. | — | — |
| Lysine | —(CH₂)₄NH₂ | Lys⁰ (K⁰) | 35° C. | 0.71 | 2.26 |
| Proline (exp)^{c} | —(CH₂)₃— | Pro (P) | 40° C. | 0.92 | 2.98 |
| Aspartic Acid | —CH₂COOH | Asp (D) | 45° C. | 0.78 | 2.57 |
| Alanine | —CH₃ | Ala (A) | 45° C. | 0.85 | 2.64 |
| Asparagine | —CH₂CONH₂ | Asn (N) | 50° C. | 0.71 | 2.29 |
| Serine | —CH₂OH | Ser (S) | 50° C. | 0.59 | 1.86 |
| Threonine | —CH(OH)CH₃ | Thr (T) | 50° C. | 0.82 | 2.60 |
| Glycine | —H | Gly (G) | 55° C. | 0.70 | 2.25 |
| Arginine | —(CH₂)₃NHC(NH)NH₂ | Arg (R) | 60° C. | — | — |
| Glutamine | —CH₂CH₂CONH₂ | Gln (Q) | 60° C. | 0.55 | 1.76 |
| Lysine | —(CH₂)₄NH³⁺ | Lys (K) | 120° C. | — | — |
| Tyrosinate | —CH₂-phenyl-O⁻ | Tyr⁻ (Y⁻) | 120° C. | 0.31 | 0.94 |
| Aspartate | —CH₂COO⁻ | Asp⁻ (D⁻) | 120° C. | — | — |
| Glutamate | —CH₂CH₂COO⁻ | Glu⁻ (E⁻) | 250° C. | — | — |

Tₜ is the inverse temperature transition for the hydrophobic folding and assembly transition, in phosphate buffered saline (0.15 N NaCl, 0.01 M phosphate) as determined by light scattering in water and by differential scanning calorimetry (“DSC”). Tₜ uses poly {ƒᵥ(GVGVP), ƒₓ(GX¹GVP)}

TABLE 1B
Tₜ-based Hydrophobicity Scale for Chemical Modifications and
Prosthetic Groups of Proteins^{a}

| Residue, X | Tₜ, linearly extrapolated to fₓ = 1 |
|---|---|
| Lys (dihydro NMeN)^{b,d} | −130° C. |
| Glu (NADH)^{c} | −30° C. |
| Lys (6-OH tetrahydro NMeN)^{b,d} | 15° C. |
| Glu (FADH₂) | 25° C. |
| Glu (AMP) | 70° C. |
| Ser (-O-SO₃H) | 80° C. |
| Thr (-O-SO₃H) | 100° C. |
| Glu (NAD)^{c} | 120° C. |
| Lys (NMeN, oxidized)^{b,d} | 120° C. |
| Glu (FAD) | 120° C. |
| Tyr (-O-SO₃H)^{e} | 140° C. |
| Tyr (-O-NO₂⁻)^{f} | 220° C. |
| Ser (PO₄^{═}) | 1000° C. |

^{a}The usual conditions are for 40 mg/ml polymer, 0.15N NaCl and 0.01M phosphate at pH 7.4.
^{b}NMeN is for N-methyl nicotinamide pendant on a lysyl side chain, i.e., N-methyl-nicotinate attached by amide linkage to the ε-NH₂ of Lys and the most hydrophobic reduced state is N-methyl-1,6-dihydronicotinamide, and the second reduced state is N-methyl-6-OH,1,4,5,6-tetrahydronicotinamide.
^{c}For the oxidized and reduced nicotinamide adenine dinucleotides, the conditions were 2.5 mg/ml polymer, 0.2M sodium bicarbonate buffer at pH 9.2.
^{d}For the oxidized and reduced N-methyl nicotinamide, the conditions were 5.0 mg/ml polymer, 0.1M potassium carbonate buffer at pH 9.5, 0.1M potassium chloride.
^{e}The pKa of polymer bound -O-SO₃H is 8.2.
^{f}The pKa of Tyr (-O-NO₂-) is 7.2.

### Free Energy Transduction By The ΔTₜ-Mechanism

As depicted in FIG. 1, fifteen pair-wise free energy conversions, involving the intensive variables of mechanical force, temperature, chemical potential, pressure, electrochemical potential, and electromagnetic radiation, become possible by controlling the value of Tₜ.

A. Tₜ-based Molecular Machines of the First Kind (Molecular Engines)

A molecular engine is a molecular machine, commonly a polymer, designed for the performance of mechanical work. Tₜ-based molecular machines of the first kind directly utilize the hydrophobic folding and assembly transition in the performance of mechanical work. All of the energy inputs in FIG. 1 that end at the mechanical force apex would be examples of different types of Tₜ-based molecular machines of the first kind. These molecular engines have been demonstrated in the configuration of γ-irradiation cross-linked protein-based polymers to form elastic bands, one millimeter thick by several millimeters in width, comprised of designed bioelastomers of compositions that lift weights as the appropriate energy inputs are introduced. The instant invention reduces the size of these molecular machines one million fold to nanometer dimensions, that is, to produce specific nanomachines.

B. Tₜ-based Molecular Machines of the Second Kind

Any two distinct functional groups, responsive to different energy inputs of FIG. 1 and each individually capable of the performance of mechanical work by altering the value of Tₜ to drive hydrophobic folding and assembly, can be coupled one to the other by being part of the same hydrophobic folding and assembly domain.

Thus Tₜ-based molecular machines of the second kind achieve free energy transductions, other than the performance of mechanical work, by also using the hydrophobic folding and assembly transition. This is Axiom 4 of Table 2, below, which contains a set of five axioms for protein engineering of protein-based polymers capable of inverse temperature transitions of hydrophobic folding and assembly, derived from the many phenomenological studies. See also, Urry, Biopolymers (Peptide Science), 47:167-178, 1998, which describes the use of the axioms in the functional design of molecular machines. While much can be achieved with the phenomenology itself, the most effective use of this capacity to control the structure and function of bioelastomers results from an understanding of the underlying physical basis.

TABLE 2

| | |
|---|---|
| Axiom 1 | The manner in which a guest amino acid residue, |
| | or chemical modification thereof, alters the temperature, |
| | Tₜ, of a hydrophobic folding and/or assembly transition is a |
| | functional measure of its hydrophobicity. A decrease in Tₜ |
| | represents increase in hydrophobicity and an increase in Tₜ |
| | represents a decrease in hydrophobicity. |
| Axiom 2 | Raising the temperature above Tₜ results in hydrophobic |
| | folding and assembly and can be used to perform useful |
| | mechanical work. e.g., of lifting weights; this is |
| | thermo-mechanical transduction. |
| Axiom 3 | At constant temperature, lowering the value of Tₜ from above |
| | to below an operating temperature, i.e., increasing the |
| | hydrophobicity by any of the many variables of |
| | TABLES 1A and 1B also results in hydrophobic folding and |
| | assembly and can be used to perform useful mechanical work |
| | of building a structure, e.g., of lifting a weight. |
| Axiom 4 | Any two distinct functional groups responsive to different |
| | variables among the many of i) temperature, ii) pressure, |
| | iii) changes in the concentrations of chemicals, iv) changes |
| | in the redox state of a biological prosthetic group, |
| | v) light elicited changes in chemical structure and other |
| | electromagnetic frequencies that alter structure, and vi) |
| | acoustic absorption, each of which could be used to |
| | alter the value of Tₜ to perform mechanical work |
| | resulting from folding and assembly, can be coupled one |
| | to the other by being part of the same hydrophobic |
| | folding and assembly domain. |
| Axiom 5 | The above energy conversions can be demonstrated |
| | to be more efficient when carried out under the influence |
| | of more hydrophobic domains. |

### The Physical Basis for the ATₜ-Mechanism (An Apolar-Polar Repulsive Free Energy Of Hydration)

A. The Tₜ-based Hydrophobicity Scale

On examination of Tables 1A and 1B, it becomes apparent that the introduction of more hydrophobicity, as in the addition of a CH₂ moiety or an aromatic moiety, lowers the value of Tₜ Since hydrophobicity is associated with hydrophobic hydration, it follows that more hydrophobic hydration lowers Tₜ and less hydrophobic hydration raises the value of Tₜ. It may also be noted in Tables 1A and 1B that the formation of charge increases the value of Tₜ. This provides the first insight that charged moieties constrained to exist along a polymer chain with hydrophobic moieties may decrease the amount of hydrophobic hydration.

B. Stretch-induced pKa Shifts, (∂μ/∂ƒ)_{n=α}<0

The first compelling argument that there exists a competition between apolar (hydrophobic) and polar (e.g., charged) species for hydration came from experimentally demonstrated stretch-induced pKa shifts. Most interestingly, as elastomeric bands of Glu-containing protein-based polymers are stretched, the pKa of the carboxyl side chain function increases, reflecting an increase in free energy of the carboxylate. With μ as the chemical potential, ƒ as the applied force and α as the degree of ionization, this is indicated by the partial at constant temperature and composition of (∂μ/∂ƒ)_{T, n=α=0.5} <0 whereas for the charge-charge repulsion case (∂μ/∂ƒ)_{T, n=α=0.5}>0.

On stretching, there is a forced unfolding of the hydrophobically folded polymer, and there is an increase in hydration, albeit hydrophobic hydration, of the elastomeric band. In spite of the added water in the elastic band, the free energy of the carboxylate state increases, as though the carboxylate were having difficulty in achieving adequate hydration. This is consistent with carboxyl destructuring water of hydrophobic hydration in the process of ionization, thus, begins an argument for a competition for hydration between apolar and polar moieties of the polymer.

C. Hydrophobic-induced pKa Shifts

If there were such a competition, then it should be possible simply to increase the hydrophobicity of a polymer containing Glu, Asp or Lys residues and effect a pKa shift, raising the pKa of carboxylates and lowering the pKa of amino groups. Indeed this is the case. Furthermore, there exists a non-linearity in which the pKa shift becomes greater for each step increase in hydrophobicity. This has been demonstrated using many different ways of increasing polymer hydrophobicity. In fact, one can routinely design at the nanometric dimension to control pKa values and for that matter to achieve all of the free energy transductions that utilize functional groups.

D. Decrease in Heat of the Inverse Temperature Transition due to Increased Degree of Ionization

By means of differential scanning calorimetry (“DSC”), it is seen that an increase in degree of ionization markedly decreases the heat of the inverse temperature transition. Two carboxylates per 100 residues can reduce the heat of the transition to one-fourth of the heat of the transition as when all Glu residues were as uncharged carboxyl moieties. The argument can be made that the endothermic heat of the transition is dominantly to destructure water of hydrophobic hydration, because there is no secondary structure change attending the inverse temperature transition of poly(GVGVP) (SEQ ID NO:1) and hydrophobic hydration is exothermic. To the extent that the endothermic heat of the transition is the heat required to destructure the amount hydrophobic hydration present before hydrophobic association, this would indicate that charge does destructure hydrophobic hydration.

E. Direct Observation of Hydrophobic Hydration by Microwave Dielectric Relaxation

The fait accompli in the argument for a competition between apolar and polar groups for hydration in polymers where the two types of moieties are constrained to coexist along a chain sequence is achieved by means of microwave dielectric relaxation data. Hydrophobic hydration exhibits a relaxation near 5 GHz, which can be quantified as a percentage of total water. This hydration increases with an increase in hydrophobic residues; it disappears on hydrophobic folding and assembly, and, most importantly for the identification of physical basis, it decreases as carboxyl ionization begins on raising the pH. Less than two carboxylates in 100 residues cause a loss of two-thirds of the hydrophobic hydration in the series of protein-based polymers studied. Thus, there has been directly demonstrated the competition for hydration between charged species and the hydrophobic side chains of Val and Phe residues. Such a competition may be described as an apolar-polar repulsive free energy of hydration.

F. Amount of Hydrophobic Hydration Determines Tₜ

Apparent above is the relationship between an increase in hydrophobic hydration and a decrease in the value of Tₜ. Comparing the value of Tₜ and the amount of hydration as the concentration of (GVGIP)₂₆₁ (SEQ ID NO:2; n=261) is increased from 40 mg/ml to 1000 mg/ml, there has been found a near linear relationship between the value of Tₜ (the onset temperature for the endothermic transition) determined by DSC and the amount of hydrophobic hydration measured by microwave dielectric relaxation. Thus, the perspective grows that the hydrophobic folding and assembly transition and the function of amphiphilic polymers are controlled by the amount of hydrophobic hydration.

### AFM-determined Single Chain Force Extension Data on a Natural Elastic Protein and on a Synthetic Bioelastomer

A. Single Chain Studies on Titin and Titin Components

By means of AFM, single chain stress/strain curves have been obtained on titin (connectin), a single 3 million Da protein chain. Most of this protein is a series of repeating sequences of 90-100 residues, but titin also contains repeating sequences of 22-26 residues for more than a 2000 residue sequence of skeletal muscle. In short, titin is a protein-based polymer; it is composed of repeating peptide sequences with known crystal structures. Titin forms a single linear molecule 4 nm in width and greater than 1000 nm in length when combed using the forces provided by a receding meniscus (See Tskhovrebova, et al., J. Mol. Biol., 265:100-106, 1997).

In the AFM experiment, one end of the single chain is attached to the cantilever tip and the other end to a surface. Force is measured as a function of chain length, and forces of a few pN/chain have been determined over an entropic elastic region (See Gaub, et al., AvH-Magazin, 71:11-18, 1998). Therefore, it is now established that entropic elasticity can occur without random chain networks and without a random, or Gaussian, distribution of end to end chain lengths. This contradicts the classical theory of rubber elasticity that has dominated teachings of entropic elasticity over the last half-century.

The 90-100 residue sequences are of two general types, so-called immunoglobulin (“Ig”) and fibronectin III (“Fn3”) domains, which form similar hydrophobically folded β-barrels that can be individually unfolded and that exhibit different critical unfolding forces arising from small differences in composition of these homologous globular protein domains. Gaub, et al., AvH-Magazin, 71:11-18, 1998 and Rief, et al., Biophys. J., 75:3008-3014, 1998, provide curves showing the unfolding of a series of Ig β-barrels and ConFn β-barrels, respectively, each with characteristic increases in length and forces for inducing unfolding. These critical unfolding forces vary with the rate of extension, reducing to a basic elastic force on very slow extension. Importantly under comparable experimental conditions, the unfolding forces range from 237 pN for Conlg constructs to 113 pN for TenFn (tenascin fibronectin) domains, and the domains unfold with characteristic increases in length.

The capacity of AFM to detect small differences in the unfolding forces and length changes on unfolding of homologous globular protein domains is relevant to the instant invention. While the sensitivity to detect differences in unfolding forces is ±10 pN, small differences in composition of 90 residue globular domains result in much larger (±100 pN) differences in the forces for unfolding. The instant invention provides, in series with an elastic protein-based polymer chain, a single hydrophobically folded globular protein with a binding site which on becoming occupied would change the equilibrium between folded and unfolded states and/or the force at which unfolding(s) would occur with attending increase(s) in length when preformed at the appropriate rate of extension.

B. Preliminary Single Chain Studies on Bioelastomers

Preparation of the bioelastomers, {(GVGVP)ₙ}m (SEQ ID NO:1; n=251; m≧2) and {(GVGIP)ₙ}ₘ (SEQ ID NO:2; n=320; m≧2), was as follows. The protein-based polymers, (GVGVP)₂₅₁ and (GVGIP)₃₂₀, were separately prepared by fermentation of the respective transformed E. coli. The gene products were purified to high level and verified as to repeat composition by 1D and 2D NMR and as to chain length by MALDI-TOF mass spectrometry. The expressed 251 mer was polymerized using EDCI such that the result would be multimers of the 251 mer and similarly for the 320 mer. That essentially all molecules were converted to higher molecular weights was verified by SDS-PAGE. The band for the 251 mer and that for the 320 mer was no longer apparent and higher molecular weight polymer was obtained, too high to penetrate significantly into the gel used to size the 251 and 320 mers. Cysteine residues were then added to both ends of the multimer, i.e., Cys {(GVGVP)ₙ}ₘCys (SEQ ID NO: 1; n=251; m≧2) and Cys{(GVGIP)ₙ}ₘCys (SEQ ID NO:2; n=320; m≧2). In short, the Cys residues were added to the ends of the chains after random chemical polymerization of the 251 or the 320 mer.

Experimental conditions for FIGS. 2A-2C were physisorption from aqueous solution (1 mg/ml) onto a gold-coated glass slide, measured in PBS using a gold coated standard silicon nitride cantilever tip. In order to limit the picking up of multiple chains, the polymers were diluted with short chains (≦5000 MW) of thiolated polyethylene glycol (“PEG”). This allowed for the bioelastomer chains to be dispersed over the surface increasing the likelihood of picking up single chains with the cantilever tip. In FIGS. 2A and 2B are shown the single chain force-extension curves for Cys{(GVGVP)ₙ}ₘCys (SEQ ID NO:1; n=251; m≧2) and Cys{(GVGIP)ₙ}ₘCys (SEQ ID NO:2; n=320; m >2). While the “length” of the force curve reflects the length of the stretched segment, not the contour length of the whole polymer molecule, the observed rupture lengths of some 700 nm would be appropriate for an n value of 2.

The curves of FIGS. 2A and 2B were fit with the worm-like chain (“WLC”) model, as used for fitting the elastic behavior of titin domains in Gaub, et al., AvH-Magazin, 71:11-18, 1998 and Rief, et al., Biophys. J., 75:3008-3014, 1998. The WLC model worked well in the range up to several 100 pN. The quality of the fit was in good agreement with the preceding results on titin, but fitted persistence lengths of 0.9 nm for GVGVP (SEQ ID NO: 1) and 1.0 nm for GVGIP (SEQ ID NO:2) are larger than for Ig or titin (0.4 nm) indicating that the bioelastomers, as single chains, are “softer” than titin.

The data of FIG. 2C for the more hydrophobic Cys{(GVGIP)ₙ}ₘCys (SEQ ID NO:2; n=320; m≧2) exhibited a hysteresis, which was not observed under very dilute conditions. This hysteresis, apparently due to extension-forced hydrophobic unfolding, may have been caused by an intermolecular aggregation rather than by the backfolding of a single strand. In the instant invention, covalent constructs of regular multi-stranded twisted filaments are utilized, where extension-forced hydrophobic unfolding is expected to produce more uniform and reproducible force-extension profiles. The instant invention also contemplates controlled backfolding of a single chain to form triple-stranded segments under the control of periodicities in the sequence, for example for intramolecular ion-pairing, and components in the media that would predispose backfolding segments to form with preferred lengths.

The nanomachines of the invention are designed by utilization of the molecular structure of bioelastomers based on the family of GVGVP (SEQ ID NO:1) type of pentamers, which allow substitution of the two Val residues with retention of the fundamental character of molecular structure and inverse temperature (phase) transition as characterized in Tables 1A and 1B. As the aggregation of phase separation and the resulting molecular structure formation are concentration dependent, this poses a challenge at the nanometer dimension to achieve the fundamental functional unit, the twisted nanofilament (described below), which forms on intra- and intermolecular hydrophobic interaction. By this means, one is able to capture at the nanometer scale all of the energy conversions to perform mechanical work, which have previously been demonstrated at the millimeter scale. This can then be used to engineer into the twisted nanofilament a single globular protein per chain to bring to bear all of the renowned specificity and selectivity of enzymes. Therefore, a change in the force-extension curve attending a change in state of functional groups along the nanofilament or on a selective interaction of an analyte with the globular protein becomes the detection of the molecular event.

Before describing the methods and compositions of the invention, it is important to have a general understanding of bioelastomers.

### The Materials

Bioelastomers have been fully characterized and described in a number of patents described below. One means of defining bioelastomers is to describe groups of peptide sequences. These materials may or may not contain ionizable amino acid residues (used for purposes described below). Specifically, these materials may be described as containing repeating units of the formula αPρΩG or αPθδ, wherein: P is a peptide-forming residue of L-proline; G is a peptide-forming residue of glycine; α is a peptide-forming residue of L-valine, L-leucine, L-isoleucine, L-phenylalanine, L-alanine or an ionizable peptide-forming residue selected from the group consisting of the residues of L-Glu, L-Asp, L-His, L-Lys, L-Tyr, and other ionizable peptide-forming L-amino acids; ρ is a peptide-forming residue of glycine or a peptide-forming residue of D-Ala, D-Glu, D-Asp, D-His, D-Lys, D-Tyr, or (optionally) other ionizable peptide-forming D-amino acids for the elastic polymeric repeats or any L-amino acid for the elastic forming repeats; Ω is a peptide-forming residue of L-valine, L-leucine, L-isoleucine, L-phenylalanine or (optionally) an ionizable peptide-forming L-amino acid or any other of the naturally occurring amino acid residues; δ is a peptide-forming residue of glycine or a peptide-forming residue of D-Glu, D-Asp, D-His, D-Lys, D-Tyr, or (optionally) another ionizable peptide-forming D-amino acid; and d is a peptide-forming residue of glycine or a peptide-forming residue of L-Glu, L-Asp, L-His, L-Lys, L-Tyr, or (optionally) another ionizable peptide-forming L-amino acid or any other of the naturally occurring amino acid residues.

Examples of these artificial or synthetic bioelastomers are described, for example, in Urry, et al., U.S. Pat. No. 4,132,746 (VPGVG and variants) (SEQ ID NO:5); Urry, U.S. Pat. Nos. 4,500,700; 4,898,926; 5,527,610; and 5,336,256, all of which describe tetrapeptide and pentapeptide repeats; Urry, U.S. Pat. No. 4,589,882 (cross-linking); Urry, et al., U.S. Pat. No. 4,783,523 (IPGVG and variants) (SEQ ID NO:6); Urry, et al., U.S. Pat. No. 4,870,055 (inclusion of hexamers); Urry, U.S. Pat. No. 5,064,430 (nonapeptide repeats); Urry, U.S. Pat. No. 5,250,516 (inverse temperature transition); Urry, et al., U.S. Pat. No. 5,854,387 (purification); and Urry, U.S. Pat. No. 5,900,405; all of which are incorporated herein by reference.

Additional examples of these artificial or synthetic bioelastomers, described for their specialized acoustic absorption properties relevant to certain nanomachine applications are described in Urry, U.S. Ser. No. 09/746,371, entitled “Acoustic Absorption Polymers And Their Methods Of Use”; all of which are incorporated herein by reference.

Numerous bioelastomers are described in the patents noted above and incorporated herein by reference. Although these patents have not been concerned with nanomachines, they provide considerable guidance on the manufacture of bioelastomers to obtain useful structural features for the uses described herein.

Another way to describe the bioelastomers suitable for use in the methods of the invention is to define them as polymers which comprise repeating elastomeric peptide monomeric units selected from the group consisting of bioelastic tetrapeptides, pentapeptides, and nonapeptides units, which comprise amino acid residues selected from the group consisting of hydrophobic amino acid and glycine residues. These monomeric units form a series of β-turns separated by dynamic bridging segments suspended between the β-turns, i.e., the monomers exist in a conformation having a β-turn of the formula:

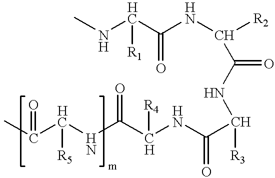

wherein R₁-R₅ represent side chains of amino acid residues 1-5, and m is 0 when the repeating unit is a tetrapeptide or 1 when the repeating unit is a pentapeptide. Nonapeptide repeating units generally consist of sequential tetra- and pentapeptides often with alternating glycines in the tetrapeptide portion. Preferred hydrophobic amino acid residues are selected from the group consisting of alanine, valine, leucine, isoleucine, proline, phenylalanine, tyrosine, tryptophan, and methionine. In many cases, the first amino acid residue of the repeating unit is a residue of valine, leucine, isoleucine, or phenylalanine; the second amino acid residue is a residue of proline; the third amino acid residue is a residue of glycine; and the fourth amino acid residue is glycine or a very hydrophobic residue such as tryptophan, phenylalanine or tyrosine, or any other of the naturally occurring amino acid residues; the fifth amino acid residue is most commonly a glycine.

Bioelastomers can be rationally designed in order to achieve the desired properties appropriate for the methods of the invention. The choice of individual amino acids from which to synthesize the elastomeric units and resulting polypeptide is unrestricted so long as the resulting structure comprises elastomeric structures with features described, for example, in U.S. Pat. Nos. 4,500,700 and 5,064,430, herein incorporated by reference, particularly the β-turn formation described above and the resulting bioclastomer maintains attributes useful for purposes intended according to the embodiments of the invention. Additionally, as the β-turns fold to form the β-spiral, acoustic absorptions become intense with dependencies on amino acid composition with features described, for example, in Urry, U.S. Ser. No. 09/746,371, supra and the resulting bioelastomer maintains acoustic absorption attributes useful for purposes intended according to the embodiments of the invention.

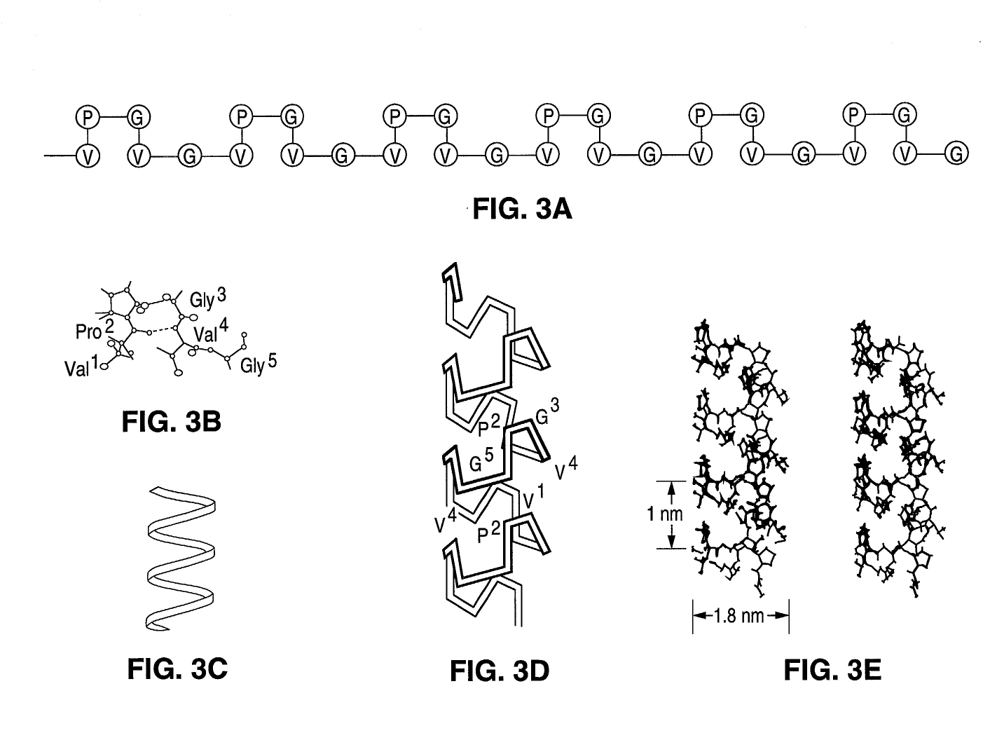

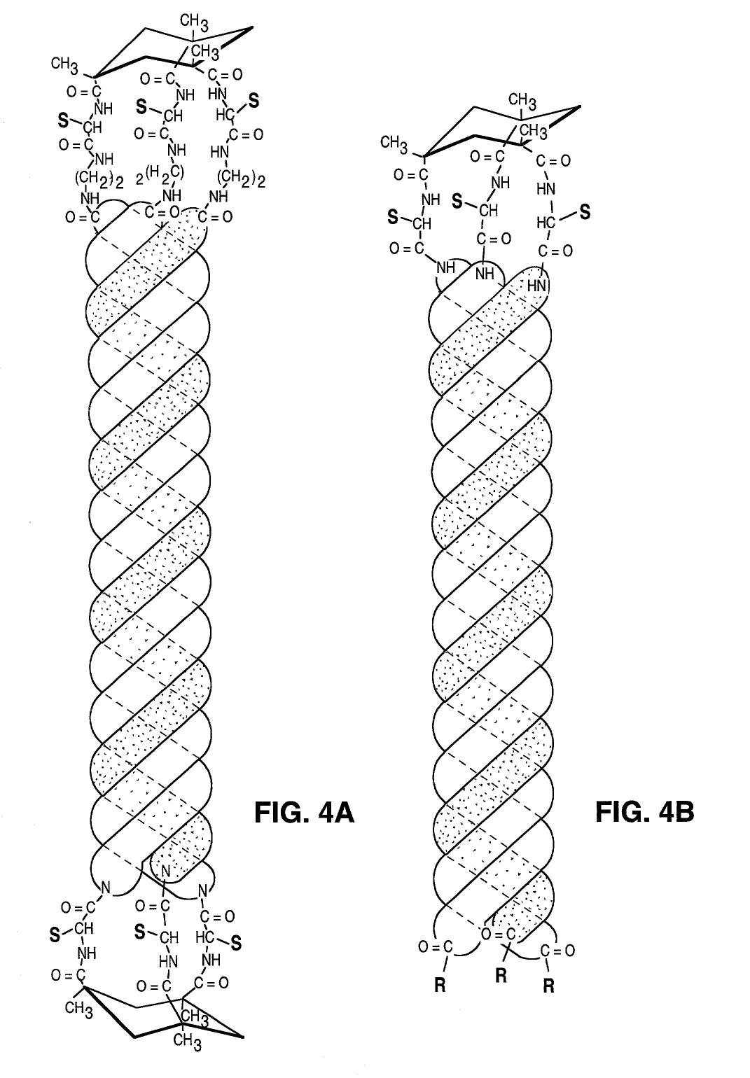

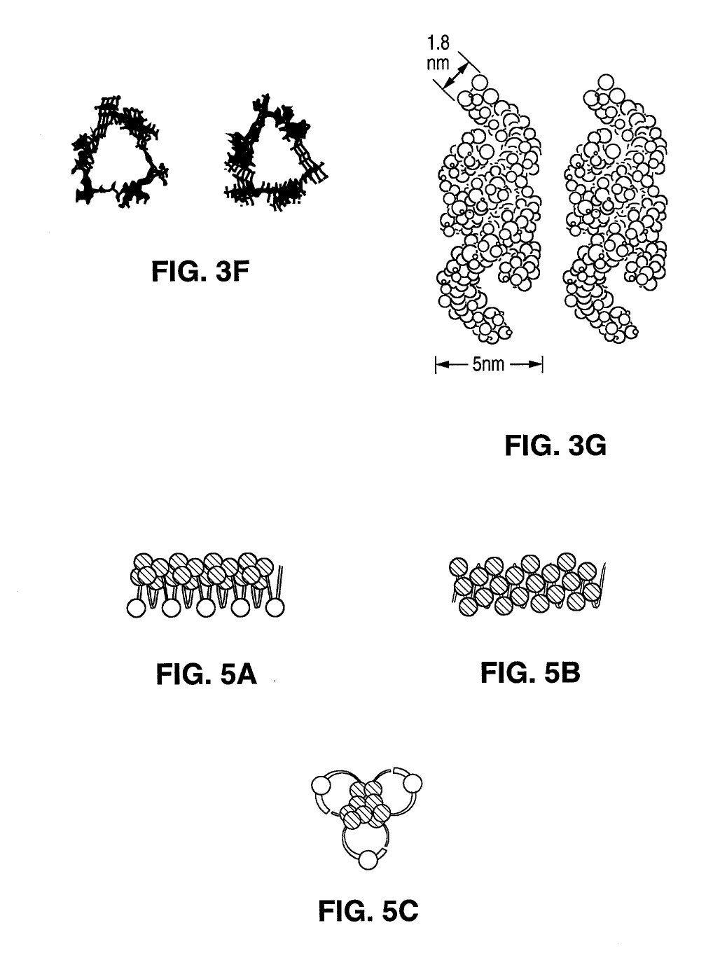

The molecular structure of poly(GVGVP) (SEQ ID NO:1) is given in FIG. 3 as a series of β-turns. The β-turns is a 10 atom hydrogen bonded ring involving the C═O of the Val residue preceding the Pro residue to the N—H of the Val residue between the two Gly residues (FIGS. 3A and 3B). The fundamental question is the relative orientation of β-turns below and above the temperature of the inverse temperature transition. Below the transition, the p-turns are essentially randomly related. Above the transition, the β-turns have hydrophobically folded into a helical array, called a P-spiral, with approximately three pentamers per turn, as seen in FIGS. 3D and 3E. Now, because of the concentration dependence of this inverse temperature transition resulting from intermolecular hydrophobic association, the β-spiral is not expected to exist alone, as in the past has often been shown schematically for simplicity. Instead, the β-spirals are to be found associated in the formation of multi-stranded twisted filaments, as seen in electron micrographs of negatively stained initial aggregates and as represented in FIG. 3F and FIG. 4.

Bioelastomers can be designed to have numerous advantages, which can be achieved by providing polymers comprised of easily obtained and coupled monomer units, e.g. amino acids, that are themselves diverse in structure and in chemical properties and are readily modified. Thus, the bioelastomer can also be present as a copolymer containing a mixture of tetrameric, pentameric or other monomeric units. Furthermore, recombinant peptide-engineering techniques can be advantageously used to produce specific peptide backbones, either in bioelastic units or non-elastic biofunctional segments.

The bioelastomers can be prepared with widely different water compositions, with a wide range of hydrophobicities, with almost any desired elastic modulus, in numerous different physical forms (e.g., sheets, gels, foams, powders, and so forth), and with a variable degree of cross-linking by selecting different amino acids for the different positions of the monomeric units and by varying the cross-linking process (e.g. chemical, enzymatic, or radiation) used to form the final product. Preparation of a variety of bioelastomers, taking into consideration these numerous aspects of polymer design, has already been described, for example in the patents referenced herein, and will therefore only be briefly described here.

The preferred bioelastomers useful in the methods of the invention are polymers comprising repeating tetrapeptide, pentapeptide and/or nonapeptide monomeric units, i.e., polytetrapeptides, polypentapeptides and polynonapeptides. Typical bioelastomers useful in this invention contain at least 5, preferably at least 10, more preferably at least 20 monomers, and even more preferably at least 100 monomers. The bioelastomers can also optionally have insertions of, for example, single amino acids between monomeric units, substitutions of one amino acid for another in an occasional monomer, or inclusion of different tetrapeptide, pentapeptide or nonapeptide sequences which can be added either in parallel or in sequence to increase strength in elastic modulus or provide some other desired characteristic. See U.S. Pat. Nos. 4,500,700 and 5,064,430. The resulting bioelastomers are thus properly known as copolymers, as they are formed from different monomeric units. A typical copolymer will preferably be a mixture of tetrapeptide and pentapeptide units, which may be the same or different, i.e., all of the tetramers may be the same or they may be different and all pentamers may be the same or they may be different. In addition, the bioelastomer can be a copolymer formed from one of the aforementioned monomeric units and a second peptide unit containing 1-100 amino acids, more typically 1-20 amino acids. Such a second peptide may have many uses such as being introduced to modify the elastic modulus, such as the hexamer, -APGVGV- (SEQ ID NO:7), described in Urry, et al., U.S. Pat. No. 4,870,055.

### Design of Single Multi-stranded Nanofilaments

The fundamental functional unit at the nanoscale dimension is the twisted filament. Because of the concentration dependence of the phase separation, it is difficult to isolate the nanofilament stage of aggregation and, of course, to do so with all chains starting and stopping together. There are many ways to form the twisted filaments, one example of which is set forth below. In order that force-extension curves can be obtained on the fundamental unit, these will need to be prepared by chemical synthesis and -SH moieties of cysteine will be positioned at each end in order to achieve firm attachment to the gold coated surfaces of cantilever tip and surface. At the amino end of the protein-based polymer chain will be adipic acid for the double-stranded filament, the Kemp tri acid for the triple-stranded filament, and EDTA for a quadruple-stranded filament. The diamine derivatives of these will be used at the carboxyl end of the bioelastomers, and attachment for closure will be carried out under extremely dilute conditions. The result will be thermally and salt-driven nanomachines without the use of fimctional side chains. The polymers to be used will be (GVGVP)₂₅₁ (SEQ ID NO: 1; n—25 1) and (GVGIP)ₙ (SEQ ID NO:2; n=320). A sketch of the triple-stranded nanofilament to be constructed using (GVGVP)₂₅₁ is given in FIGS. 4A and 4B. This structure about 60 nm in length would have the capacity to extend by a factor of about five.

Design of Acid-base- and Redox-driven Nanomachines Comprised of a Single Nanofilament

For pH-driven and redox-driven nanomachines functional side chains will be used. For those polymers containing carboxyl functional groups in the repeating basic monomer sequence, the amino end will again be attached using the multi-acids and closure at the other end will be by disulfide bridge formation using excess cysteine residues at the carboxyl end of the recombinant DNA-produced polymers. For those polymers containing amino functional groups, the carboxyl end will be used to form the initial multimer and an excess of cysteines will be used for closure of the amino end of the recombinant DNA-produced polymers. To the amino functional side chains of the formed multi-stranded filament will be added redox functionalities for production of the nanoelectromechanical system. In the case of the acid-base- and redox-driven nanomachines, the exterior of each strand of the structure of FIGS. 4A or 4B would be decorated with periodically recurring functional groups.

### Amino Acid Selection

As noted above, there are certain characteristics that are critical to the performance of the bioelastomers in the methods of the invention. However, as is apparent to one of ordinary skill in the art, there are numerous other physical properties of the bioelastomer that can be adjusted to exhibit desired characteristics, for example, viscosity, viscoelasticity, consistency, modulus of elasticity, stability, toughness, water composition, degree of hydrophobicity, physical forms and degree of cross-linking, some of which are described in detail below. Accordingly, selection of the sequence of atnino acids in a particular monomeric unit and selection of the required proportion of monomeric units can be accomplished by an empirical process that begins with determining (or looking up) the properties of known bioelastomers, making similar but different bioelastomers and measuring the physical properties as described herein and in the patents referred to above. From there, modifications can be rationally made to the selection process in order to arrive at a bioelastomer having the desired properties.

Tetramers or tetrapeptide monomeric units of particular interest include VPGG (SEQ ID NO:8) and GGX²P (SEQ ID NO:9; where X² is valine (V), phenylalanine (F) or alanine (A)).

Examples of suitable pentamers or pentapeptide monomeric units include, by way of illustration and not limitation, units having the formula GX³GX⁴P (SEQ ID NO:1O; where X3 is selected from the group consisting of valine (V), glutamic acid (E), phenylalanine (F), tyrosine (Y), lysine (K), isoleucine (I) and alanine (A); while X⁴ is selected from the group consisting of V, E, F and isoleucine (I). Examples of such formula include GVGVP, GVGIP, GVGFP, GFGFP, GFGEP, GFGIP, GEGFP, GEGVP, GKGFP, GKGVP, GEGIP, GKGIP and GYGIP. For other specifically preferred individual monomeric units and bioelastomers, see SEQ ID NOS:12, 16 and 17 and any of the patents that are herein incorporated by reference.

Particularly preferred bioelastic materials are those that contain at least one at least one repeating pentapeptide having the formula GX³GX⁴P (SEQ ID NO:10), more preferably at least one GVGVP (SEQ ID NO:1) or GVGIP (SEQ ID NO:2) pentapeptide, which can also be referred to as a-(GVGVP)ₙ-b or a-(GVGIP)ₙ-b bioelastomers, where “n” is an integer from 1 to 10,000, preferably 3 to 700, and “a” and “b” are polytetrapeptides, polypentapeptides, nonapeptides or copolymers thereof.

In particular, the specific sequences of the bioelastomers comprising the acid- base- and redox-driven nanomachines will be chosen to enhance formation of the multi-stranded nanofilament and to limit further aggregation of the chains. In one embodiment of the invention, the basic monomer sequence will be of the form (GFGFP GFGVP GAGVP GFGFP GIGVP GX⁵GVP)p, (SEQ ID NO: 11; where X⁵ is glutamic acid (E) or lysine (K), and where p is an integer from 1 to 600). As shown in FIG. 5, this sequence places the more hydrophobic Phe (F) residues in the interior of the twisted filament. On the other hand, the functional group with its carboxyl or amino, without and with attached redox group, resides on the outside of the nanofilament where the charged or oxidized states could limit further association of chains.

### Elasticity

One means of controlling the elastic modulus of bioelastomers pertains to a characteristic referred to as the transition temperature (“Tt”). A unique aspect of some bioelastomers is that they undergo an inverse temperature transition, during which a regular structure develops, unlike the random network structure of typical rubbers. This is described in detail in Urry, U.S. Pat. No. 5,250,516. At temperatures above its Tₜ, a bioelastomer associates reversibly to form a dense, water-containing viscoelastic phase, which is called the coacervate, and the solution above the coacervate is referred to as the equilibrium solution. This process of raising the temperature to form the elastomeric state results in the development of a regular structure that is a β-spiral, a loose water-containing helical structure with β-turns as spacers between turns of the helix which provides hydrophobic contacts between helical turns and with suspended peptide segments. Accordingly, the elastomeric force of these bioelastomers develops as the regular structure thereof develops. By synthesizing bioelastic materials having varying molar amounts of the constituent repeating units and by choosing a particular solvent to support the initial viscoelastic phase, it is possible to rigorously control the temperature at which the obtained bioelastomer develops elastomeric force. Maximum elastomeric force develops over a relatively narrow temperature range at temperatures spanning a range of up to about 75° C.

Accordingly, one consideration in selecting the sequence of amino acids in a particular monomeric unit and selection of the required proportion of monomeric units can be accomplished by an empirical process that begins with determining (or looking up) the properties of known bioelastomers, making similar but different bioelastomers and measuring the Tₜ and physical properties as described herein and in the patents referred to above. For example, the effect of changing the amino acid composition on the value of the transition temperature (“Tₜ”) can be determined using a hydrophobicity scale such that a rough estimate of the likely Tₜcan be obtained by summing the mean hydrophobicities of the individual amino acid residues in the monomeric units of the bioelastomer and comparing the result to the sum obtained for bioelastomers having known Tₜ. Typically, more hydrophobic residues (e.g., Ile, Phe) lower Tₜ, whereas less hydrophobic residues (e.g., Ala, Gly) and polar residues (e.g., Asp, Lys) raise Tₜ. Virtually every variable can, with the appropriate composition of the bioelastomer, change the value of Tₜ. Such variables include, among other criteria, (1) polymer concentration, (2) polymer length, (3) amino acid composition, (4) presence of salts e.g. the Hofineister (Lyotropic) Series, (5) organic solutes and solvents, (6) polymer side-chain ionization, (7) chemical modification of polymer side-chains e.g. phosphorylation, sulfation or nitration, (8) pressure e.g., as effecting aromatic residues, (9) redox state of chemical groups attached to the polymer, (10) light absorption by chemical groups attached to the polymer, and (11) side chain neutralization by ion pairing e.g., cation neutralization of anionic side chains, anion neutralization of cationic side chains, and ion-pairing between side chains.

### Backbone Modifications

The bioelastomers are composed of peptide units that form a matrix, which can be modified in a variety of ways to obtain additional properties. For example, one or more of the peptide bonds can be optionally replaced by substitute linkages such as those obtained by reduction or elimination. Thus, one or more of the —CONH— peptide linkages can be replaced with other types of linkages such as —CH₂NH—, —CH₂S—, CH₂CH—, —CH═CH— (cis and trans), —COCH₂—, —CH(OH)CH₂—, and —CH₂SO—, by methods known in the art, for example, see Spatola, A. F. (1983) in “Chemistry and Biochemistry of Amino Acids, Peptides and Proteins” (B. Weinstein, ed., Marcel Dekker, New York), p. 267 for a general review. Amino acid residues are preferred constituents of these polymer backbones. Of course, if backbone modification is made in the elastomeric units, then suitable backbone modifications are those in which the elasticity and inverse temperature transition of the polymer is maintained.

### Cross-linking

The degree of cross-linking can be controlled by selecting different amino acids for the different positions of the monomeric units and by varying the cross-linking process (e.g. chemical, enzymatic, or radiation) used to form the final product. For example, bioelastomer characteristics can be affected by cross-linking using any of various cross-linking processes, e.g., chemical, enzymatic, or irradiative. Cross-linking provides mechanical strength and rigidity to the bioelastomer, and increasing amounts of cross-linking are appropriate for increasing demands of rigidity. Cross-linking to provide one cross-link for every 200-500 repeating units is generally acceptable, with more cross-linking being permitted in less viscous bioelastomers and vice versa. Methods for cross-linking bioelastomers are known in the art, such as Urry, U.S. Pat. No. 4,589,882, which teaches enzymatic cross-linking by synthesizing block polymers having enzymatically cross-linkable units. For example, cysteine can be introduced into the bioelastomer to allow for linkage via disulfide bridges to a surface, or lysine can be introduced for enzymatic linkage to a surface, using an enzyme that cross-links for example, collagens and elastins. Another example is a bioelastomer containing one or more monomers that have a lysine (K) residue, such as GKGVP (SEQ ID NO:10; where X³ is K and X⁴ is V), which has been shown to be a substrate for the cross-linking enzyme lysyl oxidase.

Cross-linking may also be achieved by use of water soluble carbodiimides to cross-link the carboxyls of glutamic acid (Glu, E) or aspartic acid (Asp, D) on one chain to the amino function of a lysine (Lys, K) residue of another chain to form an amide. This is relevant to carboxyl or Glu-containing sequences combining with amino or Lys-containing sequences. The approach to this chemical cross-linking using water soluble carbodiimide is the following: A solution of Glu-containing bioelastomer in water (40 mg/mL) at pH 7.5 is mixed with a solution of Lys-containing bioelastomer in water (40 mg/ML) at pH 7.5. The solution is equilibrated at 2 to 3° C. above its transition temperature. A calculated quantity of EDCI and HOBt is added. The pH is adjusted to 7.5 with N-methylmorpholine and maintained above temperature with shaking for two days.

Additionally, cross-linking by irradiation is described in detail in nearly all of the patents referred to herein. For example, (GVGVP)ₙ (SEQ ID NO: 1), when prepared with n on the order of 200 and when cross-linked with 20 Mrads of γ-irradiation, forms an elastic matrix with an elastic modulus in the range of 10⁵ N/m². By variations in composition and conditions, the elastic modulus can be varied from 104 to 108 N/m2. Another such bioelastomer is X²⁰-poly(GVGVP) (Urry, et al., J. Bioactive Compatible Polym. 6:263-282 (1991)). Bioelastomers that are prepared by irradiation cross-linking are identified as, for example, “X²⁰-polyVPGVG,” which refers to a bioelastomer prepared from VPGVG pentapeptide units which has been irradiated with a 20 Mrad dose of cobalt-60 radiation to form the cross-links, thus resulting in an insoluble matrix. Cross-linked coacervates can also be obtained by much higher and lower radiation dosages, as high as 50 Mrad, but usually less than 20 Mrad, often less than 10 Mrad, and even less than 5 Mrad.

### Overall Amino Acid Composition

Considerable variations in the amino acids that are present at various locations in the resulting bioelastomer is also possible as long as the multiple β-turns with intervening suspended bridging segments are retained in order to preserve elasticity. For this reason, it is preferred that at least 50% of the polypeptide is formed from the repeating monomeric units, more preferably at least 70%, even more preferably at least 90%. Nevertheless, it is possible to prepare polypeptides in which these monomeric units are interspersed throughout a larger polypeptide that contains peptide segments designed for other purposes. Such sequences can be added covalently and sequentially or as side chains to provide for the desired function. The ratio of these other sequences to the monomer residue can range from 1:2 to 1:5000. Preferably the ratio is 1:10 to 1:100. The upper limit on the number and kind of substituents is also influenced by the ability of the bioelastomer to fold/assemble properly to attain a β-spiral in the relaxed state.

### Overall Hydrophobicity

The hydrophobicity of the overall bioelastomer (and therefore the average hydrophobicity of functional groups present in the bioelastomer) can be modified by changing the ratio of different types of monomeric units. These can be monomeric units containing a functional group undergoing the transition or other monomeric units present in the bioelastomer. For example, if the basic monomeric unit is GVGVP (SEQ ID NO: 1) and the unit undergoing transition is GX⁶GVP (SEQ ID NO:12; where X⁶ is an amino acid residue modified to have an electroresponsive side chain), either the ratio of GVGVP unit to GX⁶GVP units can be varied or a different structural unit, such as GVGIP (SEQ ID NO:2), can be included in varied amounts until the appropriate transitions temperature is achieved. Furthermore the precisely specifiable sequence of the bioelastomers allows optimal arrangement of the structural components. For example, optimal spatial proximity can be achieved by placing coupled residues adjacent to each other in the backbone (i.e., based on primary sequence) and also by positioning to provide inter-turn proximity.

A major advantage of the bioelastic polypeptides is the extent to which fine-tuning of the degree of hydrophobicity/polarity and resulting shift in the inverse temperature transition can be achieved. In addition to changes to the amino acid composition as noted above, any chemical means of changing the mean hydrophobicity of the bioelastomer, such as dephosphorylation and phosphorylation, reduction and oxidation of a redox couple, ionization and deionization, protonation and deprotonation, cleavage and ligation, amidation and deamidation, a conformational or a configurational change (e.g., cis-trans isomerization), an electrochemical change (e.g., pKa shift), emission/absorbance, or other physical change (e.g., heat energy radiation/absorbance), pressure (See U.S. Pat. No. 5,226,292), photoresponsive or electroresponsive effects, or combinations thereof, can be used to effect the Tₜ.

The hydrophobicity is easily designed by selection of appropriate amino acid residues. There is a discussion of this selection process below, as it pertains to hydrophobic characteristics of bioelastomers in general. For their specific use in the methods of the instant invention, there are three preferred residues that occur in one of more of the monomeric units making up the bioelastomer: phenylalanine, tyrosine and isoleucine. Accordingly, in one preferred embodiment of the invention, the bioelastomer comprises at least monomeric unit containing a phenylalanine or isoleucine residue. Pentamers of particular interest are charged and hydrophobically varied analogues of bioelastomers based upon the GVGVP (SEQ ID NO: 1) pentapeptide and GVGIP (SEQ ID NO:2). Accordingly, in one embodiment of the invention preferred bioelastomers contain at least one such pentamer analogue having the formula GX³GX⁴P (SEQ ID NO: 10; where X³ is V, E, F, Y or K and X⁴ is V, E, F or I).

Preferred X⁴ residues include F, such as in the pentamers GFGFP, GFGEP, GFGVP, and GFGIP, A such as in the pentamer GAGVP and GAGIP, Y such as in the pentamer GYGIP and GYGVP, E such as in the pentamer GEGIP and GEGVP, and K such as in the pentamer GKGIP and GKGVP, for example. A preferred X⁵ residue is selected from the group consisting of F and I, such as in the pentamers GAGIP, GAGFP, GVGIP, GFGIP, GVGFP, GFGFP, GEGFP and GKGFP, for example. Particularly preferred bioelastic materials are those that contain at least one GVGIP monomer, as the presence of at least one of these monomers has been shown to provide for a tougher matrix and one with a higher degree of hydrophobicity.

Examples of bioelastomers comprising one or more of these preferred monomer units include the polymers listed below, which list is intended to be exemplary and not limiting in any manner:

(GFGFP GFGVP GAGVP GFGFP GIGVP GEGVP)ₚ (SEQ ID NO:11; where X⁵ is E and p is an integer from 1 to 600)

(GAGFP GFGVP GAGVP GIGFP GFGVP GKGVP)q (SEQ ID NO:13; where q is an integer from 1 to 600)

Although the values of the integers “p” and “q” are provided above, it is understood that that is illustrative of the bioelastomers of the invention and is not intended to be limiting.

### Synthesis

In order to obtain high molecular weight bioelastomers in good yields, a number of approaches are available. Synthesis of the bioelastic repeating units is straightforward and easily accomplished by a peptide chemist or by standard methods in recombinant DNA technology and microbial fermentation. For example, organic synthesis of the bioelastomers has been described in the patents incorporated herein by reference. In particular, the synthesis and cross-linking of poly(GVGVP) have been described in U.S. Pat. No. 4,783,523. The synthesis of poly(IPGG) has been described in U.S. Pat. No. 5,250,516 and that of poly(GGAP) in US Pat. No. 5,527,610. Accordingly, the teachings of these patents can be applied to the synthesis of bioelastomers having different monomer units. When producing bioelastomers by chemical synthesis, care should be taken to avoid impurities, because small levels of impurities can result in termination of the polymerization process or in racemerization that can alter the physical properties of the resulting bioelastomer, but there are otherwise no particular problems of synthesis. Peptide unit purity is important in obtaining a material with suitable physical properties since, for example, small changes in the preparation of the bioelastomers can result in a Tₜ that varies as much as 15° C. The solution of this potential problem is simply to purify the components used to prepare the peptide.

The bioelastomer can be prepared as a homopolymer or a copolymer. Either random or block copolymers prepared from at least two of the monomeric units are useful in the methods of the present invention but are less preferred when an equivalent homopolymer has the desired physical properties, simply because of the greater complexity of synthesis. Irrespective of how the bioelastomers are synthesized, these can further be derivatized, if desired. For example, electroresponsive side chains can be incorporated into the bioelastomer as described in Urry, U.S. Pat. No. 5,900,405 (electrical exposure).

The bioelastomers can also be prepared using genetic engineering techniques. Using this approach, a gene encoding the desired peptide sequence is constructed, artificially inserted into, and then translated in a host organism. The organism can be prokaryotic, e.g., bacterial, or eukaryotic, e.g., yeast or plants. Techniques are known in the art of molecular biology to manipulate genetic information (i.e., DNA sequences) for effective gene expression in an appropriate host organism (see, for example, Sambrook et al., Molecular Cloning: A Laboratory Manual, Second Edition, Cold Spring Harbor, New York (1989)) and include the use of enzymes capable of cleaving, joining, copying or otherwise modifying polynucleotides. In addition, vectors allowing the introduction of this information into the host organism in a suitable manner for expression are known in the art. A detailed example of the production of poly-VPGVG is set out in McPherson, et al., Biotechnol. Prog. 8:347-352 (1992), and McPherson, et al., Protein Expression and Purification 7:51-57 (1996), publications arising from the laboratory of the present inventor. These publications can be used as guidance for genetic-based production of any of the materials used in the present invention. Bioelastomers with as many as 2000 amino acid residues have been expressed in good quantity by appropriate E. coli strains. For example, expression of (GVGVP)ₙ (SEQ ID NO:1; where n=121) has occurred at levels of 80% of E. coli cell volume. Accordingly, many of the bioelastomers suited for use in this methods of this invention can be synthesized at low cost by use of E. coli, transformed to produced (GVGIP)ₙ (SEQ ID NO:2; n=260), the expression vector which is then modified for co-expression of an easily measured expression product.

In general, single-stranded oligonucleotides encoding select portions of the desired bioelastomer are chemically synthesized by a commercial source. These oligonucleotides are then are annealed at their 3′ ends through a complimentary region, and extended to the full length, double stranded basic gene fragment by a high fidelity thermostable DNA polymerase. The resulting gene fragment is digested by the restriction enzyme BamHI before its insertion into the cloning vector pUC 118 through ligation. After the transformation of the cloning host E. coli DH5aF′, the positive clones are recovered from the selection plates and the plasmid DNA from each clone is isolated for the screening analysis. The resulting plasmids are digested with BamHI and separated on an agarose gel. Clones are selected as candidates for subsequent sequence verification. Once the sequence is verified, this clone is used as a source of the desired bioelastomer for the ensuing gene construction. To construct a concatemer (multimer) gene, a large amount of the monomer gene fragments are prepared after digesting the monomer gene containing plasmid with the restriction enzyme. The resulting monomer gene fragments are then concatenated (ligated) in the presence of N- and C-terminal adaptors, which provide the cloning sites for the subsequent manipulation in different vectors. The resulting concatenation products, consisting of multimer genes of varying chain length, are ligated into pUC118 and introduced into E coli. The possible clones on the selection plates are screened and the positive clones identified after a series of digestion with different restriction enzymes.

By using recombinant DNA technology, the cost of production of bioelastomers can be competitive with synthetic, organic polymers and with natural materials that need extensive purification. Producers of industrial proteins have demonstrated that costs can be reduced significantly for biologically produced proteins. cost is of particular importance when producing bioelastomers for use as acoustic absorbers, due to the large quantities needed.

The bioelastic polypeptide can be purified, for example, from cultures grown in fermentation reactors or from organic syntheses, by its ability to undergo an inverse temperature transition. Purification using the inverse temperature transitional properties of the protein-based polymers is preferred with genetically engineered polymers expressed in microbial systems as even endotoxin levels have been demonstrated to be particularly reduced using this method. See Urry, et al., J. Biomater. Sci. Polymer Edn. 9:1015-1048 (1998).

### Methods of Use

The following examples describe specific aspects of the invention to illustrate the invention and provide a description of the present method for those of skill in the art. The examples should not be construed as limiting the invention, as the examples merely provide specific methodology useful in understanding and practice of the invention.

### EXAMPLES

The preparation of the bioelastomers utilizes gene construction, development of an expression system, fermentation and purification as described for the particular examples given below. As for gene construction, the basic monomer genes were designed to have appropriate cohesive ends comprising an appropriate restriction site sequence (selected using standard techniques, such as consideration of the restriction sites present in the vector to be used for expression). Polymerization of the basic gene was carried out through the compatible sticky ends generated by restriction endonuclease digestion, followed by subsequent ligation using DNA ligase to form multimers of the basic gene. This protocol was used successfully to produce each of the monomer gene sequences. The monomers were then concatemerized (polymerized) to form multimer genes with many different numbers of repeats, and many of the multimer genes have been expressed at high levels as will be briefly reported below.

### Example 1

### The Preparation of Nanomachines

A. Preparation of the Bioelastomers by Recombinant DNA Technology

The bioelastomers are prepared using recombinant DNA technology, by the preparation of basic monomer genes, their concatemerized into multimer genes, introduction into E. coli and expression at high levels. The general approach is described in McPherson, et al., Protein Expression and Purification 7:51-57, 1996.

B. Gene Sequences

Specific gene designs are required for (GFGFP GFGVP GAGVP GFGFP GIGVP GEGVP)ₚ (SEQ ID NO: 11; where X⁵ is E and p is 50) and (GAGFP GFGVP GAGVP GIGFP GFGVP GKGVP)q (SEQ ID NO:13; where q is 45).

The nucleotide sequence (SEQ ID NO: 14) for (GFGFP GFGVP GAGVP GFGFP GIGVP GEGVP)ₚ (SEQ ID NO: 11, where p is 2) is given below as an example.

```
      BamHI PflMI
gaggatcca GGC TTT GGT TTC CCG GGT TTC GGC GTT CCG GGC GCT GGT GTA CCG
                      G F G F P G F G V P G A G V P
                    GGT TTC CCC TTT CCG GGT ATC GGT GTT CCG GGC GAA CGT GTG CCA
                      G F G F P G I G V P G E G V P
                    GGA TTC GGC TTC CCG GGC TTT GGT GTT CCG GGT GCA GGC GTA CCG
                      G F G F P G F G V P G A G V P
                    GGT TTC GGT TTC CCG GGC ATT GGC GTT CCG GGT GAA GGT GTA CCA
                      G F G F P G I G V P G E G V P
                    ggctttggatccag (SEQ ID NO:14)
                    PflM-I BamH-I (SEQ ID NO:11, where p is 2)
```

C. Insertion of Globular Module Between Long Runs of Bioelastomer

A globular module can be inserted between long runs of bioelastomer by using the GRGDSP-containing Fn3 domain (SEQ ID NO:15), but to replace the GRGDSP cell attachment sequence with a kinase recognition site such as RGYSLG (SEQ ID NO:16). Alternately, the small globular protein, lysozyme, with its RGYSLG kinase recognition site can be used. The amino acid sequence of the Fn3 domain with the RGYSLG kinase recognition site, Fn3:RGYSLG, is as follows:

| | |
|---|---|
| VSDVPRDLEVVAATPTSLLISWDAPAVTVRYYRITYGETGG | (SEQ ID |
| NSPVQEFTVPGSKSTATISGLKPGVDYTITVYAVTRGYSLG | NO:57) |
| ASSKPISINYRTEIDKP | |

The gene encoding the globular Fn3 domain was constructed such that it could be concatenated with monomer genes encoding a bioelastomer, for instance (GVGIP)lo (SEQ ID NO:2; n=10). In addition, it could be concatenated with itself to produce multiple repeats of the Fn3: RGYSLG. The monomer Fn3:RGYSLG gene was constructed in two “half” genes using the following synthetic oligonucleotide, where N denotes the amino-terminal half and C denotes the carboxyl-terminal half:

Fn3Dom-N1 66 mer (SEQ ID NO:49)
GGA ACT GGT TGC TGC AAC TCC GAC TTC TCT GCT
GAT CTC CTG GGA CGC TCC AGC AGT TAC CGT ACG
Fn3Dom-N2 66 mer (SEQ ID NO:50)
GAA CCG GAG AGT TGC CAC CAG TCT CGC CGT AGG
TGA TGC GAT AGT AAC GTA CGG TAA CTG CTG GAG
Fn3Dom-N3 67 mer (SEQ ID NO:51)
gag gat cca ggc gtt ggc att ccg GTT TCT GAC GTA
CCG CGT GAT CTG GAA GTG GTT GCT GCA ACT C
Fn3Dom-N4 64 mer (SEQ ID NO:52)
ctg gat cca acg cct ggg ata cct aca ccc atg
gtc tGA ATT CCT GAA CCG GAG AGT TGC CAC C
Fn3Dom-C1 73 mer (SEQ ID NO:53)
CCA GGC AGC AAG TCC ACT GCG ACC ATT TCT GGT CTG
AAA
CCG GGT GTT GAC TAC ACC ATC ACT GTG TAC GCT G
Fn3Dom-C2 73 mer (SEQ ID NO:54)
GTA GTT AAT GGA GAT CGG TTT GCT GGA AGC ACC CAG
AGA
ATA GCC ACG AGT TAC AGC GTA CAC AGT GAT GGT G
Fn3Dom-C3 73 mer (SEQ ID NO:55)
gag gat cca ggc gtt ggc att ccg taa gct ttc tcg
aga
gaG AAT TCA CCG TAC CAG GCA GCA AGT CCA CTG C
Fn3Dom-C4 72 mer (SEQ ID NO:56)
ctg gat cca acg cct ggg ata cct aca ccC GGT TTG
TCG ATT TCG GTA CGG TAG TTA ATG GAG ATC GGT TTG

Specifically, for each “half” gene, two oligonucleotides were annealed through a region of homology at their 3′ ends. These ends were extended with thermostable DNA polymerase and free deoxy-nucleotides in a thermal cycler to give a double-stranded DNA fragment. An aliquot of this reaction product was combined in a similar reaction mixture containing two other oligonucleotides, which were homologous at their 3′ ends with the 3′ ends of the double-stranded fragments of the first two oligonucleotides. The resulting double-stranded products of these reactions are given below with their encoded amino acid sequences indicated. The Fn3 :RGYSLG encoding DNA sequence is indicated by capital letters.

```
7 Fn3Dom-N:
        PflMI
BamHI |
    | |
gaggatccaggcgttggcattccgGTTTCTGACGTACCGCGTGATCTGGAAGTGGTT
            P G V G I P V S D V P R D L E V V
ctcctaggtccgcaaccgtaaggcCAAAGACTGCATGGCGCACTAGACCTTCACCAA
    | |
    3 7
GCTGCAACTCCGACTTCTCTGCTGATCTCCTGGGACGCTCCAGCAGTTACCGTACGT
A A T P T S L L I S W D A P A V T V R
CGACGTTGAGGCTGAAGAGACGACTAGAGGACCCTGCGAGGTCGTCAATGGCATGCA
                                                                                          EcoRI
                                                                                                |
TACTATCGCATCACCTACGGCGAGACTGGTGGCAACTCTCCGGTTCAGGAATTCaga
Y Y R I T Y G E T G G N S P V Q E F
ATGATAGCGTAGTGGATGCCGCTCTGACCACCGTTGAGAGGCCAAGTCCTTAAGtct
                                                                                                |
                                                                                              163
NcoI PflMI BamHI
| | |
ccatgggtgtaggtatcccaggcgttggatccag (SEQ ID NO:58)
    M G V G I P G V (SEQ ID NO:59)
ggtacccacatccatagggtccgcaacctaggtc (SEQ ID NO:60)
| | | |
172 189 198 205
7 Fn3Dom-C:
        PflMI XhoI
BamHI HindIII | EcoRI
    | | | | |
gaggatccaggcgttggcattccgtaagctttctcgagagaGAATTCACCGTA
            P G V G I P * E F T V
ctcctaggtccgcaaccgtaaggcattcgaaagagctctctCTTAAGTGGCAT
    | | | | |
    3 7 26 33 42
CCAGGCAGCAAGTCCACTGCGACCATTTCTGGTCTGAAACCGGGTGTTGACTAC
P G S K S T A T I S G L K P G V D Y
GGTCCGTCGTTCAGGTGACGCTGGTAAAGACCAGACTTTGGCCCACAACTGATG
ACCATCACTGTGTACGCTGTAACTCGTGGCTATTCTCTGGGTGCTTCCAGCAAA
T I T V Y A V T R G Y S L G A S S K
TGGTAGTGACACATGCGACATTGAGCACCGATAAGAGACCCACGAAGGTCGTTT
                                                                                                PflMI
                                                                                                      |
CCGATCTCCATTAACTACCGTACCGAAATCGACAAACCGggtgtaggtatccc
P I S I N Y R T E I D K P G V G I P
GGCTAGAGGTAATTGATGGCATGGCTTTAGCTGTTTGGCCcacatccataggg
                                                                                                      |
                                                                                                  213
          BamHI
              |
aggcgttggatccag (SEQ ID NO:61)
G V (SEQ ID NO:62)
tccgcaacctaggtc (SEQ ID NO:63)
              |
              222
```

Each of these Fn3:RGYSLG “half” genes was cloned into a multipurpose cloning vector, for instance pUCI 18, utilizing the terminal BamHI sites and was verified by DNA sequence analysis. Subsequently, they were each released from the cloning vector by cleavage at PflMI and could be concatenated independently with the Pf1MI digested monomer bioelastomer gene. Each “half” gene could be mixed, with ligase, with the bioelastomer gene at a ratio that favors the desired number of bioelastomer repeats per number of half gene repeats.

A concatenation ligation reaction containing, for instance, the Fn3Dom-N monomer gene with the (GVGIP)₁₀ monomer gene, can give a concatemer product of one to multiple repeats of the (GVGIP)₁₀ gene flanked on both ends by one to multiple repeats of the Fn3Dom-N gene. If this concatemer gene, (Fn3Dom-N)ₓ((GVGVIP)₁₀)_{y}(Fn3Dom-N)_{z} [where x, y and z are greater than or equal to 1], is digested with NcoI and EcoRi it will result in the Fn3Dom-N half gene with y copies of the (GVGVP)₁₀ gene appended to its 5′ end. Similarly, if the ligation reaction is performed with the Fn3Dom-C half gene and (GVGIP)₁₀monomer resulting in (Fn3Dom-C)_{q}((GVGVIP)₁₀)ᵣ(Fn3Dom-C)ₛ [where q, r and s are greater than or equal to 1] and the product is digested with EcoRI and HindIII, it will result in the Fn3Dom-C half gene with r copies of the (GVGIP)₁₀ gene appended to its 3′ end. These two concatenation products can be ligated through their EcoRI ends to produce a complete Fn3:RGYSLG gene sequence with y and r copies of (GVGIP)₁₀ at its 5′ and 3′ ends, respectively. In addition, the Fn3Dom-N and -C “half” genes each contribute a GVGIP coding sequence at the NcoI and HindIII ends, as well as a start and stop codon at these ends, respectively. The resulting gene encodes GVGIP((GVGIP)₁₀)_{y}Fn3:RGYSLG((GVGIP)₁₀)ᵣGVGIP and can be cloned into, for instance, an expression vector via the NcoI and HindIII ends.

D. Chemical Preparation of the Multi-stranded Twisted Filaments

For those bioelastomers with carboxyl side chains, the tri-cysteine-derivatized Kemp tri-acid, for example, will be used to attach the strands by their amino ends. The following is given as one possible approach. The other end of the bioelastomer chain will be cross-linked by disulfides arising from cysteine residues added by the adaptor oligonucleotides used during concatenation. The number and locations of Cys residues at the carboxyl termini will be chosen to achieve both intermolecular disulfide cross-bridges and attachment to the gold surface by having unreacted -SH functional groups. Analogously for bioelastomers with amino side chains, the tri-ethylenediamine, tri-cysteine derivative of the Kemp tri-acid, for example, will be used to attach the chains by their carboxyl ends. Similarly, the sequence of the adaptor oligonucleotides will be used to insert the cysteines for inter-chain disulfide bridge cross-links as desired at the amino terminus, while retaining residual cysteine residues with -SH functional groups for attachment to the gold coated surfaces.

### Example 2

### AFM Characterization of the Nanomachines

The atomic force microscope (“AFM”) is a high resolution scanning-probe microscope which can be operated in liquid environments. Next to its most popular application as an imaging device, the AFM has emerged as a force sensing device on the nanometer and piconewton scale which can thus be used to perform mechanical experiments with single chain molecules. Both aspects of modem AFM technology will be employed for the characterization of the twisted filament nanomachines.

A. Single Filament Force-extension Curves

For the elastic characterization of single filaments, their force extension curves will be investigated using AFM-based single molecule force spectroscopy. This uses specialized instrumentation, which allows for optimized spanning and stretching of individual polymer chains between the tip of and AFM-cantilever and a substrate surface. When renouncing a lateral scanning of the sample, the z-range of the piezo translator is 8 (m and the piezo extension is controlled by a built-in strain gauge. With this instrument, a precision of a few Angstroms in controlling he cantilever position can be achieved with a simultaneous force resolution in the piconewton (“pN”) range (the thermal noise dues to cantilever oscillations is below 10 pN, and can be further improved by filtering to ΔF≈3 pN). The nominal spring constants of cantilevers used in the elastic experiments on polypeptides were approximately 10 mN/m, the resonance frequencies of the cantilevers in aqueous environments were typically on the order of 1 kHz. Prior to the first approach of the AFM tip to the surface, the spring constants of each lever are individually calibrated by measuring the amplitude of its thermal oscillations. The typical time scale to record a single stretching trace is 1 millisecond to 100 seconds.

Typically, the experiments are conducted at room temperature in liquid environments such as pure MilliQ water or an aqueous electrolyte buffer. The instrument's sample cell allows for the exchange of liquid, such that conditions of salt or pH can be varied during an experiment while a polymer chain is held between the AFM tip and the substrate. In addition, the gold-coated tip and substrate are electrically conducting, and can thus be used as electrodes for the stimulation of redox processes occurring at the molecular level. Finally, experiments can also be conducted to allow for coupling an external optical stimulus (laser light) into the system. Further experimental details are described in Oesterhelt, et al., New Journal of Physics 1:6.1-6.11, 1999 and Clausen-Schaumann, et al., Biophys. J. 78:1997-2007,2000.

The fixation of the bioelastomers being evaluated to an AFM tip and to the substrate can be achieved either by specific ligand-receptor binding, by covalent bonds, as well as by specific thiols on gold) or even non-specific adsorption. In preliminary work, individual single strands of the bioelastomers Cys{(GVGVP)₂₅₁}ₘCys (SEQ ID NO:1; n=251; m >2) and Cys{(GVGIP)₃₂₀}ₘCys (SEQ ID NO:2; n—320; m≧2) were adsorbed onto gold-coated glass slides from a dilute aqueous solution at 4° C. (i.e., below the Tₜ of the bioelastomers). Thiol-terminated polyethylene glycol (“PEG”, Polysciences) of low average molecular weight (MW 5000 g/mol, i.e., average contour length of (30 nm) was used as a coadsorbent to realize a dilute dispersion of the bioelastomers art the surface.

In the AFM experiment, a gold coated Si₃N₄ cantilever tip (Microlevers, Park Scientific instruments, Sunnyvale, Calif.) was brought into close contact with the interfacial polymer layer by manual control for 10-30 seconds. Upon retraction of the cantilever, one or several polymer strands adhered to the tip, and the resulting force-distance profile was measured via the deflection of the AFM cantilever spring using optical lever detection. By the use of the coadsorbed PEG, the non-specific adhesion between the AFM tip and the gold substrate is completely suppressed due to steric repulsion resulting form the interfacial polymer layer. Moreover, as the average contour length of the coadsorbed PEG is an order of magnitude shorter than the length of the bioelastomer strands being evaluated, this strategy provides a high probability of picking up single bioelastomer strands in the experiment. The much shorter PEG chains are easily recognized by their characteristic force-extension profile. Finally, the specific binding between thiol end-groups of the bioelastomers and the gold-coated tip allows for repeatedly measuring the elastic characteristics of one individual bioelastomer strand up to nanonewton forces.

Preliminary examination of individual bioelastomer strands, as reported in FIG. 2, proves the feasibility of the planned AFM characterization of bioelastomers which provides the basis for the controlled mechanical manipulation of single multi-stranded twisted filaments.

B. Imaging of the Twisted Filament Nanomachine

The elastic characterization of the twisted multi-stranded filaments will be supplemented by AFM imaging. This will allow for the investigation and better control of the structure of the bioelastomer nanomachines at the substrate surface.

The imaging of the twisted filament nanomachines will be carried out at ambient temperature (25° C.) using a commercially available instrument (Nanoscope IIIa, Digital Instruments). The samples can be imaged in air as well as in an appropriate buffer solution. For the latter purpose, the sample will be mounted in a fluid cell which can be sealed to prevent evaporation of the buffer solution in long-term studies. A piezo scanner with a maximum range of 15 (m and silicon nitride cantilevers with integrated oxide sharpened tips (also from Digital Instruments, nominal spring constant 30 mN/m) will be used. Tapping-mode will allow minimization of lateral forces between the tip and the substrate. (See Hansma, et al., Applied Physics Letters 64:1738, 1994 and Putman, et al., Applied Physics Letters 64:2454, 1994).

C. Utilizing the Acoustic Absorption Properties of these Bioelastic Polymers

As a marked advance over the tapping-mode, the cantilevers of the AFM instrument can be made to vibrate to cover the frequency range of the acoustic absorption of these bioelastic polymers. (See Tamayo, et al., Applied Physics Letters, 77(4):582-584, 2000). When used in the AFM scanning mode with the vibration parallel to the substrate, this provides for the frequency dependence of G′, the shear storage modulus and G″, the shear loss modulus, which is equivalent to the absorption per unit volume of FIG. 7. (See Antognozzi, et al, Ultramicroscopy 86:223-232, 2001). This provides for a means of monitoring structural changes resulting from passing through the inverse temperature transition of hydrophobic folding and assembly. If the vibration were perpendicular to the substrate and the AFM operated in the force-extension mode, then this would provide for the characterization of the component of the mechanical resonance that is parallel to the twisted filament axis. In one embodiment of the invention, the bioelastic twisted filaments are designed such that changes in the structural state upon introduction of an energy input to be detected can be sensed by changes in the intensity and frequency of the mechanical resonance.

The following bioelastic polymers, described in Urry, U.S. Ser. No. 09/746,371, supra, are particularly suited to provide for control of frequency ranges and absorption intensities:

| | | |
|---|---|---|
| Polymer I: | (GVGVP)₂₅₁ | (SEQ ID NO:1; n = 251). |
| Polymer II: | (GVGIP)₂₆₀ | (SEQ ID NO:2; n = 260). |
| Polymer III: | (GVGVP GVGFP GEGFP GVGVP GVGFP GFGFP)ₙ (GVGVP) | (SEQ ID NO:20, n = 32). |
| Polymer IV: | (GVGVP GVGFP GEGFP GVGVP GVGFP GVGFP)ₙ (GVGVP) | (SEQ ID NO:21; n = 41). |
| Polymer V: | (GVGVP GVGVP GEGVP GVGVP GVGFP GFGFP)ₙ (GVGVP) | (SEQ ID NO:22; n = 39). |
| Polymer VI: | (GVGVP GVGFP GEGFP GVGVP GVGVP GVGVP)ₙ (GVGVP) | (SEQ ID NO:23; n = 40). |
| Polymer VII: | (GVGVP GVGVP GEGVP GVGVP GVGVP GVGVP)ₙ (GVGVP) | (SEQ ID NO:24; n = 36). |
| Polymer VIII: | (GVGIP GFGEP GEGFP GVGVP GFGFP GFGIP GVGIP GFGEP | (SEQ ID NO:25; n = 20). |
| | GEGFP GVGVP GFGFP GFGIP)ₙ (GVGVP) | |
| Polymer IX: | (GVGVP GVGFP GKGFP GVGVP GVGFP GFGFP)ₙ (GVGVP) | (SEQ ID NO:26; n = 21). |
| Polymer X: | (GVGVP GVGFP GKGFP GVGVP GVGFP GVGFP)ₙ (GVGVP) | (SEQ ID NO:27; n = 21). |
| Polymer XI: | (GVGVP GVGVP GKGVP GVGVP GVGFP GFGFP)ₙ (GVGVP) | (SEQ ID NO:28; n = 22). |
| Polymer XII: | (GVGVP GVGFP GKGFP GVGVP GVGVP GVGVP)ₙ (GVGVP) | (SEQ ID NO:29; n = 25). |
| Polymer XIII: | (GVGVP GVGVP GKGVP GVGVP GVGVP GVGVP)ₙ (GVGVP) | (SEQ ID NO:30; n = 35). |
| Polymer XIV: | (GVGVP GVGFP GEGFP GVGVP GVGFP GKGVP)ₙ (GVGVP) | (SEQ ID NO:31; n = 21). |
| Polymer XV: | (GVGVP GVGFP GEGFP GVGVP GVGVP GKGVP)ₙ (GVGVP) | (SEQ ID NO:32; n = 21). |

Although the values of the integer “n” are provided above, it is understood that that is illustrative of the polymers of the invention and is not intended to be limiting. Polymers having repeating units such as those described for Polymers I-XV can be designed to have n values within the range of 1 to 5000. The following polymers are also useful (n is an integer from about 1 to 5000):

| | | |
|---|---|---|
| Polymer I′ | [(GVGIP GEGIP GVGIP)₃]ₙ | SEQ ID NO: 33 |
| Polymer II′ | [(GVGIP GVGIP GEGIP | SEQ ID NO: 34 |
| | GVGIP GVGIP GVGIP)]ₙ | |
| Polymer III′ | [(GEGIP GVGIP GEGIP | SEQ ID NO: 35 |
| | GVGIP GVGIP GVGIP)]ₙ | |
| Polymer IV′ | [(GVGIP GKGIP GVGIP)3]ₙ | SEQ ID NO: 36 |
| Polymer V′ | [(GVGIP GVGIP GKGIP | SEQ ID NO: 37 |
| | GVGIP GVGIP GVGIP)]ₙ | |
| Polymer VI′ | [(GKGIP GVGIP GKGIP | SEQ ID NO: 38 |
| | GVGIP GVGIP GVGIP)]ₙ | |
| Polymer VII′ | [(GVGIP)₂₁(GVGIP)]ₙ | SEQ ID NO: 39 |
| Polymer VIII′ | [(GVGIP)₂₁(GY{SO₄^{═}}GIP)]ₙ | SEQ ID NO: 40 |
| Polymer IX′ | [(GVGIP)₁₁(GYGIP)]ₙ | SEQ ID NO: 41 |
| Polymer X′ | [(GVGIP)₁₁(GY{SO₄^{═}}GIP)]ₙ | SEQ ID NO: 42 |
| Polymer XI′ | [(GVGIP)₈(GYGIP)]ₙ | SEQ ID NO: 43 |
| Polymer XII′ | [(GVGIP)₈(GY{SO₄^{═}}GIP)]ₙ | SEQ ID NO: 44 |
| Polymer XIII′ | [(GVGIP)₅(GYGIP)]ₙ | SEQ ID NO: 45 |
| Polymer XIV′ | [(GVGIP)₅(GY{SO₄^{═}}GIP)]ₙ | SEQ ID NO: 46 |
| Polymer XV′ | [(GVGIP)₂(GYGIP)]ₙ | SEQ ID NO: 47 |
| Polymer XVI′ | [(GVGIP)₂(GY{SO₄^{═}}GIP)]ₙ | SEQ ID NO: 48 |

### Example 3

### Construction and Characterization of Multi-stranded Nanofilaments and Nanochemomechanical Systems

Double-, triple- and quadruple-stranded twisted filaments can be prepared and their AFM characterizations conducted. In addition, nanochemomechanical systems (“NCMS”) can be produced, which will utilize sequence of Phe and Glu residues for directing and limiting aggregation to nanofilament formation. The carboxyl function will be used to drive contraction/relaxation, with the sequence schematically indicated in FIGS. 5A-5C, i.e., (GEGVP GFGFP GFGVP GAGVP GFGFP GIGVP)ᵣ (SEQ ID NO: 17; where r is an integer from 1 to 500).

In the process of determining the pH-dependence of force development and of length changes, the AFM studies in addition to the general characterizations can evaluate (∂f/∂μ), and (≠l/≠μ)_{f} for most the suitable experimental conditions to follow nanofilament chemomechanical transduction.

### Example 4

### Construction and Characterization of Multi-stranded Nanofilaments for Nanoelectromechanical Systems

The amino function and attached redox functions can be attached and used to drive contraction/relaxation, with the sequence similar to that indicated in FIGS. 5A-5C, but with Lys as the more polar (vinegar-like) residue, i.e., (GKGVP GAGFP GFGVP GAGVP GIGFP GFGVP)s (SEQ ID NO:18; where s is an integer from 1 to 500). The pH-dependence of force development and of length changes will be determined with the Lys-containing function, and the effect of the oxidative state of the redox function on force development and on length changes will be determined with the redox-containing function. Any of the redox couples of Table 1B can be used, for example, N-methyl nicotinamide (NMeN).

### Example 5

### Incorporation of Hydrophobically Folded Globular Protein

The gene is made for the incorporation of a hydrophobically folded globular protein in series with the bioelastomer to provide a model, for example, for a functional biosensor for toxic gas, DNT or TNT interaction to drive toward relaxation (unfolding) of the hydrophobically folded globular protein. The physical basis for this approach is the apolar-polar repulsive free energy of hydration, which is the competition for hydration between polar and hydrophobic moieties constrained to coexist along a polymer chain. The binding of a polar species to a hydrophobically folded globular protein module destructures part of the hydrophobic hydration for the unfolded state, thereby shifting the equilibrium in the direction of the unfolded state. In general, this can be used to detect unfolding of all or a component of the globular protein wherein the contour length of the globular module containing nanofilament, as measured by the force-extension curve, would increase by the added length of the unfolded chains.

By way of example, on having designed for the correct operating temperature range, it has been shown that the binding of one phosphate per 300 residues of (GVGIP) (SEQ ID NO:2) causes complete hydrophobic unfolding. A properly positioned kinase recognition site in a titin-like β-barrel would, on phosphorylation, be expected to cause the β-barrel simply to unfold and increase the contour length by an amount characteristic of the globular unit, as seen with the globular units of titin. Therein lies the capacity to detect interaction of a polar species at a site on a hydrophobically folded globular protein or any molecular addition that alters the intensity of the hydrophobic domain. In addition, such an observation using the AFM approach constitutes another demonstration of the mechanism of free energy transduction with all of the potential that such a capacity would yield.

Nitration of tyrosine residues markedly raises the value of Tₜ (See Table 1B). Accordingly, the binding of DNT to a suitable site of globular unit, such as a titin-like β-barrel, can be expected to destabilize the hydrophobically folded globular unit with attending changes in the nanofilament force-extension curve. Alternatively, enzymes, globular proteins, that reduce DNT would have their redox prosthetic group oxidized. As the oxidation of prosthetic group changes the value of Tₜ, this effect can be used for AFM detection of analytes of interest.

The simplest test construct can be bioelastomer(“BE”)-globular protein(“GP”)-bioelastomer (“BE”). The two identical bioelastomer components would exhibit a simple monotonic curve like those of FIGS. 2A and 2B, and the single globular protein would exhibit its characteristic sawtooth pattern, which could be a single tooth or it could be a more complex pattern in which one or more domains unfolded separately. The key to the use of such a construct as a sensor would be that the binding of the analyte should alter the pattern recognized as characteristic of the free globular protein. The initial demonstration could be a kinase recognition site.

Two different approaches could be considered. One would use a small globular protein which naturally contains a kinase recognition site such as lysozyme with the sequence RGYSLG (SEQ ID NO:16). The second approach would replace a cell attachment site, such as the GRGDSP (SEQ ID NO: 15) cell attachment site, with a kinase recognition site such as RGYSLG. As noted above, it has already been shown that the RGYSLG sequence in a poly(GVGIP) (SEQ ID NO:2) sequence dramatically shifts the value of Tₜ and drives hydrophobic unfolding while removal drives hydrophobic folding Pattanaik, et al, Biochem. Biophys. Res. Comm., 178:539-545, 1991.

FIG. 6 gives a schematic set of force-extension curves with curve A being a simple single strand of bioelastomer (“BE”) as in FIGS. 2A and 2B. Curve B of FIG. 6 shows the force-extension profile for BE-GP-BE, where GP is a globular protein like lysozyme with two folding domains, one weaker and a second requiring a greater force to unfold. Curve C is a hypothetical example where binding of analyte, e.g., in the case of lysozyme a phosphorylation, which completely unfolds the weaker domain and lowers the force required to unfold the stronger domain.

All publications and patent applications mentioned in this specification are herein incorporated by reference to the same extent as if each individual publication or patent application was specifically and individually indicated to be incorporated by reference at the location where cited. The invention now being fully described, it will be apparent to one of ordinary skill in the art that many changes and modifications can be made thereto without departing from the spirit or scope of the invention.

What is claimed is:

1. A nanomachine comprising a bioelastomer having repeating peptide monomeric units selected from the group consisting of nonapeptide, pentapeptide and tetrapeptide monomeric units, wherein said monomeric units form a series of β-turns separated by dynamic bridging segments suspended between said β-turns.

2. The nanomachine of claim 1 wherein the bioelastomer is in the form of nanoparticles.

3. The nanomachine of claim 1 wherein the bioelastomer is in the form of multi-stranded nanofilaments.

4. The nanomachine of claim 3 wherein each nanofilament has a di-, tri-, or tetra-acid at each end.

5. The nanomachine of claim 4 wherein said acid is selected from the group consisting of adipic acid, Kemp triacid and ethylenediaminetetraacetic acid.

6. The nanomachine of claim 1 wherein the nanomachine is formed by a single bioelastomer chain folding back on itself with the forced unfolding causing a peaking or an increase in force of the force-extension profile.

7. The nanomachine of claim 6 wherein the single chain has a di-, tri-, or tetra-acid at each end.

8. The nanomachine of claim 7 wherein said acid is selected from the group consisting of adipic acid, Kemp triacid and ethylenediaminetetraacetic acid.

9. The nanomachine of claim 1 which is a nanochemomechanical system.

10. The nanomachine of claim 1 which is a nanoelectromechanical system.

11. The nanomachine of claim 1 which is a nanobaromechanical system.

12. The nanomachine of claim 1 which is a nanothermomechanical system.

13. The nanomachine of claim 1 which is a nanophotomechanical system.

14. The nanomachine of claim 1 which is a biosensor.

15. The nanomachine of claim 1 wherein the bioelastomers are less than 10, 000 amino acid residues in length.

16. The nanomachine of claim 1 wherein the bioelastomer has a di-, tri-, or tetra-acid at each end and is strung between a cantilever and a substrate.

17. The nanomachine of claim 16 wherein the tip of the cantilever and the surface of the substrate are attached by cysteinyl sulfurs at the acid ends.

18. The nanomachine of claim 16 wherein the tip of the cantilever and the surface of the substrate are attached by an amino or carboxyl functional group that has replaced the cysteinyl sulfur moiety at the acid ends.

19. The nanomachine of claim 16 wherein the cantilever senses the vibrational energy absorbed by the bioelastomer.

20. The nanomachine of claim 1 wherein one serine residue is phosphorylated per every 30-500 amino acid residues in said bioelastomer.

21. The nanomachine of claim 1 wherein the carboxyl side chains of two amino acid residues are ionized per every 20-200 amino acid residues in said bioelastomer.

22. The nanomachine of claim 1 wherein there are two amino acid residues per every 20-200 amino acid residues in said bioelastomer that are attached to redox functionalities.

23. The nanomachine of claim 1 wherein the bioelastomer comprises an elastomeric polypentapeptide.

24. The nanomachine of claim 23 wherein the bioelastomer comprises at least one pentapeptide having the formula GX³GX⁴P (SEQ ID NO:10), where X³ is selected from the group consisting of valine (V), glutamic acid (E), phenylalanine (F), tyrosine (Y), lysine (K), isoleucine (I) and alanine (A); and X⁴ is selected from the group consisting of V, E, F and isoleucine (I).

25. The nanomachine of claim 24 wherein at least one of said pentapeptide monomeric units is GVGVP (SEQ ID NO:1) or GVGIP (SEQ ID NO:2).

26. The nanomachine of claim 1 wherein said bioelastomer is cross-linked.

27. The nanomachine of claim 1 wherein said bioelastomer comprises a block or random copolymer comprising at least two of said monomeric units.

28. The biosensor of claim 1 wherein said bioelastomer further comprises at least one cell attachment site.

29. The biosensor of claim 28 wherein said cell attachment site has the formula GRGDSP (SEQ ID NO:15).

30. The biosensor of claim 1 wherein said bioelastomer further comprises at least one kinase recognition site.

31. The biosensor of claim 30 wherein said kinase recognition site has the formula RGYSLG (SEQ ID NO:16).

32. The nanomachine of claim 1 wherein said bioelastomer is selected from the group consisting of SEQ ID NO:1; SEQ ID NO:2; SEQ ID NO:20; SEQ ID NO:21; SEQ ID NO:22; SEQ ID NO:23; SEQ ID NO:24; SEQ ID NO:25; SEQ ID NO:26; SEQ ID NO:27; SEQ ID NO:28; SEQ ID NO:29; SEQ ID NO:30; SEQ ID NO:31; SEQ ID NO:32; SEQ ID NO:33; SEQ ID NO:34; SEQ ID NO:35; SEQ ID NO:36; SEQ ID NO:37; SEQ ID NO:38; SEQ ID NO:39; SEQ ID NO:40; SEQ ID NO:41; SEQ ID NO:42; SEQ ID NO:43; SEQ ID NO:44; SEQ ID NO:45; SEQ ID NO:46; SEQ ID NO:47; and SEQ ID NO:48.

33. The nanomachine of claim 1 which is able to sense energy input or providing energy output over a frequency range of about 10 to 10¹⁵ cycles per second.

34. The nanomachine of claim 33 wherein the frequency range is about 10-10⁵ cycles per second.

35. The nanomachine of claim 34 wherein the bioelastomer is encoded by a nucleotide sequence selected from the group consisting of SEQ ID NO:49, SEQ ID NO:50, SEQ ID NO:51, SEQ ID NO:52, SEQ ID NO:53, SEQ ID NO:54, SEQ ID NO:55 and SEQ ID NO:56.

36. The nanomachine of claim 33 wherein the frequency range is about 10²-10⁴ cycles per second.

37. A biosensor comprising a bioelastomer having repeating peptide monomeric units selected from the group consisting of nonapeptide, pentapeptide and tetrapeptide monomeric units, wherein said monomeric units form a series of β-turns separated by dynamic bridging segments suspended between said β-turns.

38. The biosensor of claim 37 wherein the bioelastomer is in the form of nanoparticles.

39. The biosensor of claim 37 wherein the bioelastomer is in the form of multi-stranded nanofilaments.

40. The nanomachine of claim 37 wherein the biosensor is formed by a single bioelastomer chain folding back on itself with the forced unfolding causing a peaking or an increase in force of the force-extension profile.

41. The biosensor of claim 37 which is useful for detecting the presence of chemical species.

42. The biosensor of claim 37 wherein one serine residue is phosphorylated per every 30-500 amino acid residues in said bioelastomer.

43. The biosensor of claim 37 wherein the carboxyl side chains of two amino acid residues are ionized per every 20-200 amino acid residues in said bioelastomer.

44. The biosensor of claim 37 wherein there are two amino acid residues per every 20-200 amino acid residues in said bioelastomer that are attached to redox functionalities.

45. The biosensor of claim 37 wherein the bioelastomer comprises an elastomeric polypentapeptide.

46. The biosensor of claim 45 wherein the bioelastomer comprises at least one pentapeptide having the formula GX³GX⁴P (SEQ ID NO:10), where X³ is selected from the group consisting of valine (V), glutamic acid (E), phenylalanine (F), tyrosine (Y), lysine (K), isoleucine (I) and alanine (A); and X⁴ is selected from the group consisting of V, E, F and isoleucine (I).

47. The biosensor of claim 46 wherein at least one of said pentapeptide monomeric units is GVGVP (SEQ ID NO:1) or GVGIP (SEQ ID NO:2).

48. The biosensor of claim 37 wherein said bioelastomer is cross-linked.

49. The biosensor of claim 37 wherein said bioelastomer comprises a block or random copolymer comprising at least two of said monomeric units.

50. The biosensor of claim 37 wherein said bioelastomer further comprises at least one cell attachment site.

51. The biosensor of claim 50 wherein said cell attachment site has the formula GRGDSP (SEQ ID NO:15).

52. The biosensor of claim 37 wherein said bioelastomer further comprises at least one kinase recognition site.

53. The biosensor of claim 52 wherein said kinase recognition site has the formula RGYSLG (SEQ ID NO:16).

54. The biosensor of claim 37 which further comprises a single globular domain containing a binding site in series with said bioelastomer, wherein binding of an analyte at the binding site causes the hydrophobically folded globular domain to unfold at a different force level.

55. The biosensor of claim 54 wherein the globular domain contains an enzyme site that, upon phosphorylation, causes the hydrophobically folded globular domain to unfold at a lower force level.

56. The biosensor of claim 37 wherein said bioelastomer is selected from the group consisting of SEQ ID NO:1; SEQ ID NO:2; SEQ ID NO:20; SEQ ID NO:21; SEQ ID NO:22; SEQ ID NO:23; SEQ ID NO:24; SEQ ID NO:25; SEQ ID NO:26; SEQ ID NO:27; SEQ ID NO:28; SEQ ID NO:29; SEQ ID NO:30; SEQ ID NO:31; SEQ ID NO:32; SEQ ID NO:33; SEQ ID NO:34; SEQ ID NO:35; SEQ ID NO:36; SEQ ID NO:37; SEQ ID NO:38; SEQ ID NO:39; SEQ ID NO:40; SEQ ID NO:41; SEQ ID NO:42; SEQ ID NO:43; SEQ ID NO:44; SEQ ID NO:45; SEQ ID NO:46; SEQ ID NO:47; and SEQ ID NO:48.
